SEQUENCE LISTING (20 sequences)

SEQ ID NO: 1 - protein, 378 aa, homo sapiens
MPGWGRESVGTHSIRPRGTWASNGPPRVRSGRGGPALLPASGQCRVLGRRSCRACWGASTCRWAPHGEGRRPRDRALPGTSGWRVPDTNGRKPPDTNARNPRQATTEGDYLPSDPAAGVRKKRSPVCSDWSRLFDVTDSTFDRATRRRGACGSRGGNFPSPRGGSGVASLERAESSSTEPAKAIKPIDRKSVHQICSGPVVPSLSTAVKELVENSLDAGATNIDLKLKDYGVDLIEVSGNGCGVEEENFEGLTLKHHTSKIQEFADLPQVETFGFRGEALSSLCALSDVTISTCHVSAKVGTRLVFDHYGKIIQKTPYPHPRGMTVSVKQLFSTLPVHHKEFQRNIKKKRACFPFAFCRDCQFPEASPAMLPVQPAEL

SEQ ID NO: 2 - DNA, 1164 bp, homo sapiens
atgccggggtggggccgcgaatcggttgggacgcactctatccggcctaggggcacctgggccagcaacgggccgccgcgcgtgcgcagtgggcgggggggccccgcgctcctacctgcaagtggccagtgccgagtgctgggccgccgctcctgccgtgcatgttggggagccagtacatgcaggtgggctccacacggagaggggcgccgaccccgtgatagggctttacctggtacatcggggtggcgcgtgccagacaccaacggtcggaaaccgccagacaccaacgctcggaatccacgccaggccacgacggagggcgactacctcccttctgaccctgctgctggcgttcggaaaaaacgcagtccggtgtgctctgattggtccaggctctttgacgtcacggactcgacctttgacagagccactaggaggcggggcgcctgtgggagccgtggagggaactttcccagtccccgaggcggatccggtgttgcatccttggagcgagctgagagctcgagtacagaacctgctaaggccatcaaacctattgatcggaagtcagtccatcagatttgctctgggccggtggtaccgagtctaagcactgcggtgaaggagttagtagaaaacagtctggatgctggtgccactaatattgatctaaagcttaaggactatggagtggatctcattgaagtttcaggcaatggatgtggggtagaagaagaaaacttcgaaggcttaactctgaaacatcacacatctaagattcaagagtttgccgacctacctcaggttgaaacttttggctttcggggggaagctctgagctcactttgtgcactgagtgatgtcaccatttctacctgccatgtatcggcgaaggttgggactcgactggtgtttgatcactatgggaaaatcatccagaaaaccccctacccccaccccagagggatgacagtcagtgtgaagcagttattttctacgctacctgtgcaccataaagaatttcaaaggaatattaagaagaaacgtgcctgcttccccttcgccttctgccgtgattgtcagtttcctgaggcctccccagccatgcttcctgtacagcctgcagaactgtgagtcaattaaacctgttttcttcataaa

SEQ ID NO: 3 - protein, 782 aa, homo sapiens
MGKRDRADRDKKKSRKRHYEDEEDDEEDAPGNDPQEAVPSAAGKQVDESGTKVDEYGAKDYRLQMPLKDDHTSRPLWVAPDGHIFLEAFSPVYKYAQDFLVAIAEPVCRPTHVHEYKLTAYSLYAAVSVGLQTSDITEYLRKLSKTGVPDGIMQFIKLCTVSYGKVKLVLKHNRYFVESCHPDVIQHLLQDPVIRECRLRNSEGEATELITETFTSKSAISKTAESSGGPSTSRVTDPQGKSDIPMDLFDFYEQMDKDEEEEEETQTVSFEVKQEMIEELQKRCIHLEYPLLAEYDFRNDSVNPDINIDLKPTAVLRPYQEKSLRKMFGNGRARSGVIVLPCGAGKSLVGVTAACTVRKRCLVLGNSAVSVEQWKAQFKMWSTIDDSQICRFTSDAKDKPIGCSVAISTYSMLGHTTKRSWEAERVMEWLKTQEWGLMILDEVHTIPAKMFRRVLTIVQAHCKLGLTATLVREDDKIVDLNFLIGPKLYEANWMELQNNGYIAKVQCAEVWCPMSPEFYREYVAIKTKKRILLYTMNPNKFRACQFLIKFHERRNDKIIVFADNVFALKEYAIRLNKPYIYGPTSQGERMQILQNFKHNPKINTIFISKVGDTSFDLPEANVLIQISSHGGSRRQEAQRLGRVLRAKKGMVAEEYNAFFYSLVSQDTQEMAYSTKRQRFLVDQGYSFKVITKLAGMEEEDLAFSTKEEQQQLLQKVLAATDLDAEEEVVAGEFGSRSSQASRRFGTMSSMSGADDTVYMEYHSSRSKAPSKHVHPLFKRFRK

SEQ ID NO: 4 - DNA, 2751 bp, homo sapiens
gggagcttccggattgagccggaagtccccccagagcggatgccgcggcgggcctgtgggagcggggtcatcttctctctgctgctgtagctgccatgggcaaaagagaccgagcggaccgcgacaagaagaaatccaggaagcggcactatgaggatgaagaggatgatgaagaggacgccccggggaacgaccctcaggaagcggttccctcggcggcggggaagcaggtggatgagtcaggcaccaaagtggatgaatatggagccaaggactacaggctgcaaatgccgctgaaggacgaccacacctccaggcccctctgggtggctcccgatggccatatcttcttggaagccttctctccagtttacaaatatgcccaagacttcttggtggctattgcagagccagtgtgccgaccaacccatgtgcatgagtacaaactaactgcctactccttgtatgcagctgtcagcgttgggctgcaaaccagtgacatcaccgagtacctcaggaagctcagcaagactggagtccctgatggaattatgcagtttattaagttgtgtactgtcagctatggaaaagtcaagctggtcttgaagcacaacagatacttcgttgaaagttgccaccctgatgtaatccagcatcttctccaggaccccgtgatccgagaatgccgcttaagaaactctgaaggggaggccactgagctcatcacagagactttcacaagcaaatctgccatttctaagactgctgaaagcagtggtgggccctccacttcccgagtgacagatccacagggtaaatctgacatccccatggacctgtttgacttctatgagcaaatggacaaggatgaagaagaagaagaagagacacagacagtgtcttttgaagtcaagcaggaaatgattgaggaactccagaaacgttgcatccacctggagtaccctctgttggcagaatatgacttccggaatgattctgtcaaccctgatatcaacattgacctaaagcccacagctgtcctcagaccctatcaggagaagagcttgcgaaagatgtttggaaacgggcgtgcacgttcgggggtcattgttcttccctgcggtgctggaaagtccctggttggtgtgactgctgcatgcactgtcagaaaacgctgtctggtgctgggcaactcagctgtttctgtggagcagtggaaagcccagttcaagatgtggtccaccattgacgacagccagatctgccggttcacctccgatgccaaggacaagcccatcggctgctccgttgccattagcacctactccatgctgggccacaccaccaaaaggtcctgggaggccgagcgagtcatggagtggctcaagacccaggagtggggcctcatgatcctggatgaagtgcacaccataccagccaagatgttccgaagggtgctcaccatcgtgcaggcccactgtaagctgggtttgactgcgaccctcgtccgcgaagatgacaaaattgtggatttaaattttctgattgggcctaagctctacgaagccaactggatggagctgcagaataatggctacatcgccaaagtccagtgtgctgaggtctggtgccctatgtctcctgaattttaccgggaatatgtggcaatcaaaaccaagaaacgaatcttgctgtacaccatgaaccccaacaaatttagagcttgccagtttctgatcaagtttcatgaaaggaggaatgacaagattattgtctttgctgacaatgtgtttgccctaaaggaatatgccattcgactgaacaaaccctatatctacggacctacgtctcagggggaaaggatgcaaattctccagaatttcaagcacaaccccaaaattaacaccatcttcatatccaaggtaggtgacacttcgtttgatctgccggaagcaaatgtcctcattcagatctcatcccatggtggctccaggcgtcaggaagcccaaaggctagggcgggtgcttcgagctaaaaaagggatggttgcagaagagtacaatgcctttttctactcactggtatcccaggacacacaggaaatggcttactcaaccaagcggcagagattcttggtagatcaaggttatagcttcaaggtgatcacgaaactcgctggcatggaggaggaagacttggcgttttcgacaaaagaagagcaacagcagctcttacagaaagtcctggcagccactgacctggatgccgaggaggaggtggtggctggggaatttggctccagatccagccaggcatctcggcgctttggcaccatgagttctatgtctggggccgacgacactgtgtacatggagtaccactcatcgcggagcaaggcgcccagcaaacatgtacacccgctcttcaagcgctttaggaaatgatgcttaggcagggtacttcgttcaagaccggcgcttggcacccttgttggaaagggattttcagcataacattttccttccacctctttgaccttccctccagcgttggccaaattgtgctgaggaagatgcatcaagggcttggctgtgccttcataggtcatctagggttttataaaggaggaggagacaatattttttcaaactttttggggagtggggtcatttctgtatataaaaaatgttaatatttaaggtgtatttatgttaccgttctgaataaacagaatggaccattgaaccagta

SEQ ID NO: 5 - protein, 318 aa, homo sapiens
MPKRGKKGAVAEDGDELRTEPEAKKSKTAAKKNDKEAAGEGPALYEDPPDQKTSPSGKPATLKICSWNVDGLRAWIKKKGLDWVKEEAPDILCLQETKCSENKLPAELQELPGLSHQYWSAPSDKEGYSGVGLLSRQCPLKVSYGIGDEEHDQEGRVIVAEFDSFVLVTAYVPNAGRGLVRLEYRQRWDEAFRKFLKGLASRKPLVLCGDLNVAHEEIDLRNPKGNKKNAGFTPQERQGFGELLQAVPLADSFRHLYPNTPYAYTFWTYMMNARSKNVGWRLDYFLLSHSLLPALCDSKIRSKALGSDHCPITLYLAL

SEQ ID NO: 6 - DNA, 1574 bp, homo sapiens
cagacagaccaatcacgcgcattcttcggccacgacaagcgcgcctctgatcacgtgaccaggtccgctacccacgtgggggctcagcgtgcacccttctttgtgctcgggttaggaggagctaggctgccatcgggccggtgcagatacggggttgctcttttgctcataagaggggcttcgctggcagtctgaacggcaagcttgagtcaggacccttaattaagatcctcaattggctggagggcagatctcgcgagtagggcaacgcggtaaaaatattgcttcggtgggtgacgcggtacagctgcccaagggcgttcgtaacgggaatgccgaagcgtgggaaaaagggagcggtggcggaagacggggatgagctcaggacagagccagaggccaagaagagtaagacggccgcaaagaaaaatgacaaagaggcagcaggagagggcccagccctgtatgaggaccccccagatcagaaaacctcacccagtggcaaacctgccacactcaagatctgctcttggaatgtggatgggcttcgagcctggattaagaagaaaggattagattgggtaaaggaagaagccccagatatactgtgccttcaagagaccaaatgttcagagaacaaactaccagctgaacttcaggagctgcctggactctctcatcaatactggtcagctccttcggacaaggaagggtacagtggcgtgggcctgctttcccgccagtgcccactcaaagtttcttacggcataggcgatgaggagcatgatcaggaaggccgggtgattgtggctgaatttgactcgtttgtgctggtaacagcatatgtacctaatgcaggccgaggtctggtacgactggagtaccggcagcgctgggatgaagcctttcgcaagttcctgaagggcctggcttcccgaaagccccttgtgctgtgtggagacctcaatgtggcacatgaagaaattgaccttcgcaaccccaaggggaacaaaaagaatgctggcttcacgccacaagagcgccaaggcttcggggaattactgcaggctgtgccactggctgacagctttaggcacctctaccccaacacaccctatgcctacaccttttggacttatatgatgaatgctcgatccaagaatgttggttggcgccttgattactttttgttgtcccactctctgttacctgcattgtgtgacagcaagatccgttccaaggccctcggcagtgatcactgtcctatcaccctatacctagcactgtgacaccacccctaaatcactttgagcctgggaaataagccccctcaactaccattccttctttaaacactcttcagagaaatctgcattctatttctcatgtataaaactaggaatcctccaaccaggctcctgtgatagagttcttttaagcccaagattttttatttgagggttttttgttttttaaaaaaaaattgaacaaagactactaatgactttgtttgaattatccacatgaaaataaagagccatagtttcaaaaaaaaaaaaaaaaaaaaaaaaaaa

SEQ ID NO: 7 - protein, 715 aa, homo sapiens
MELADVGAAASSQGVHDQVLPTPNASSRVIVHVDLDCFYAQVEMISNPELKDKPLGVQQKYLVVTCNYEARKLGVKKLMNVRDAKEKCPQLVLVNGEDLTRYREMSYKVTELLEEFSPVVERLGFDENFVDLTEMVEKRLQQLQSDELSAVTVSGHVYNNQSINLLDVLHIRLLVGSQIAAEMREAMYNQLGLTGCAGVASNKLLAKLVSGVFKPNQQTVLLPESCQHLIHSLNHIKEIPGIGYKTAKCLEALGINSVRDLQTFSPKILEKELGISVAQRIQKLSFGEDNSPVILSGPPQSFSEEDSFKKCSSEVEAKNKIEELLASLLNRVCQDGRKPHTVRLIIRRYSSEKHYGRESRQCPIPSHVIQKLGTGNYDVMTPMVDILMKLFRNMVNVKMPFHLTLLSVCFCNLKALNTAKKGLIDYYLMPSLSTTSRSGKHSFKMKDTHMEDFPKDKETNRDFLPSGRIESTRTRESPLDTTNFSKEKDINEFPLCSLPEGVDQEVFKQLPVDIQEEILSGKSREKFQGKGSVSCPLHASRGVLSFFSKKQMQDIPINPRDHLSSSKQVSSVSPCEPGTSGFNSSSSSYMSSQKDYSYYLDNRLKDERISQGPKEPQGFHFTNSNPAVSAFHSFPNLQSEQLFSRNHTTDSHKQTVATDSHEGLTENREPDSVDEKITFPSDIDPQVFYELPEAVQKELLAEWKRTGSDFHIGHK

SEQ ID NO: 8 - DNA, 2484 bp, homo sapiens
gctgggggtggagccggaggaggaaggcggcggcgacgacgaggaagacgccgaggcctgggccatggaactggcggacgtgggggcggcagccagctcgcagggagttcatgatcaagtgttgcccacaccaaatgcttcatccagagtcatagtacatgtggatctggattgcttttatgcacaagtagaaatgatctcaaatccagagctaaaagacaaacctttaggggttcaacagaaatatttggtggttacctgcaactatgaagctaggaaacttggagttaagaaacttatgaatgtcagagatgcaaaagaaaagtgtccacagttggtattagttaatggagaagacctgacccgctacagagaaatgtcttataaggttacagaattactggaagaatttagtccagttgttgagagacttggatttgatgaaaattttgtggatctaacagaaatggttgagaagagactacagcagctgcaaagtgatgaactttctgcggtgactgtgtcgggtcatgtatacaataatcagtctataaacctgcttgacgtcttgcacatcagactacttgttggatctcagattgcagcagagatgcgggaagccatgtataatcagttggggctcactggctgtgctggagtggcttctaataaactgttggcaaaattagtttctggtgtctttaaaccaaatcaacaaacagtcttattacctgaaagttgtcaacatcttattcatagtttgaatcacataaaggaaatacctggtattggctataaaactgccaaatgtcttgaagcactgggtatcaatagtgtgcgtgatctccaaaccttttcacccaaaattttagaaaaagaattaggaatttcagttgctcagcgtatccaaaagctcagttttggagaggataactcccctgtgatactctcaggaccacctcagtcctttagtgaagaagattcatttaaaaaatgttcatctgaagttgaagctaaaaataagattgaagaactacttgctagtcttttaaacagagtatgccaagatggaaggaagcctcatacagtgagattaataatccgtcggtattcctctgagaagcactatggtcgtgagagtcgtcagtgccctattccttcacatgtaattcagaaattagggacaggaaattatgatgtgatgaccccaatggttgatatacttatgaaactttttcgaaatatggtgaatgtgaagatgccatttcaccttacccttctaagtgtgtgcttctgcaaccttaaagcactaaatactgctaagaaagggcttattgattattatttaatgccatcattatcaactacttcacgctctggcaagcacagttttaaaatgaaagacactcatatggaagattttcccaaagacaaagaaacaaaccgggatttcctaccaagtggaagaattgaaagtacaagaactagggagtctccactagataccacaaatttttctaaagaaaaagacattaatgaattcccactctgttcacttcctgaaggtgttgaccaagaagtcttcaagcagcttccagtagatattcaagaagaaatcctttctggaaaatctagggaaaaatttcaagggaaaggaagtgtgagttgtccattacatgcctctagaggagtattatctttcttttctaaaaaacaaatgcaagatattcccataaatcctagagatcatttatccagtagcaaacaggtatcctctgtatctccttgtgaaccgggaacatcaggctttaatagcagtagttcttcttacatgtctagccaaaaggattattcatattatttagataatagattaaaagatgaacgaataagtcaaggacctaaagaacctcaaggattccactttacaaattcaaaccctgctgtgtctgcttttcattcatttccaaacttgcagagtgagcaacttttctccagaaaccacactacagatagccataagcaaacagtagcaacagactctcatgaaggacttacagaaaatagagagccagattctgttgatgagaaaattactttcccttctgacattgatcctcaagttttctatgaactaccagaagcagtacaaaaggaactgctggcagagtggaagagaacaggatcagatttccacattggacataaataagcatattcagcaaaaaggtctgaaaagcaagggaataccattattttcggattagcggtttattaagctcttctatattaaacactaatagatattcaataacggagtaaactgttccagataaagcaagaatagttgcaagaagtaaattctggcacaaagcgtaaaaatataacagaagaaataatgtaaaatactatcttttatgtctaaagccattttatattacttttcaataaaaagaatatcatggtcaaaaaaaaaaaaaaaaa

SEQ ID NO: 9 - protein, 551 aa, homo sapiens
MAAPVRLGRKRPLPACPNPLFVRWLTEWRDEATRSRHRTRFVFQKALRSLRRYPLPLRSGKEAKILQHFGDGLCRMLDERLQRHRTSGGDHAPDSPSGENSPAPQGRLAEVQDSSMPVPAQPKAGGSGSYWPARHSGARVILLVLYREHLNPNGHHFLTKEELLQRCAQKSPRVAPGSAPPWPALRSLLHRNLVLRTHQPARYSLTPEGLELAQKLAESEGLSLLNVGIGPKEPPGEETAVPGAASAELASEAGVQQQPLELRPGEYRVLLCVDIGETRGGGHRPELLRELQRLHVTHTVRKLHVGDFVWVAQETNPRDPANPGELVLDHIVERKRLDDLCSSIIDGRFREQKFRLKRCGLERRVYLVEEHGSVHNLSLPESTLLQAVTNTQVIDGFFVKRTADIKESAAYLALLTRGLQRLYQGHTLRSRPWGTPGNPESGAMTSPNPLCSLLTFSDFNAGAIKNKAQSVREVFARQLMQVRGVSGEKAAALVDRYSTPASLLAAYDACATPKEQETLLSTIKCGRLQRNLGPALSRTLSQLYCSYGPLT

SEQ ID NO: 10 - DNA, 2343 bp, homo sapiens
gtctcaaaggctggctggagtggagccaaaggaaaagatcgttagagacagcgcccctgaccaaccacttagagcagcgcaggggtgggagggcggccgcaggctctcctctcgttagtgccccctgtgtttggggccccgtgatctcaacggtcctgccctcggtctccctcttcccccgccccgccctgggccaggtgttcgaatcccgactccagaactggcggcgtcccagtcccgcgggcgtggagcgccggaggacccgccctcgggctcatggcggccccggtccgcctgggccggaagcgcccgctgcctgcctgtcccaacccgctcttcgttcgctggctgaccgagtggcgggacgaggcgacccgcagcaggcaccgcacgcgcttcgtatttcagaaggcgctgcgttccctccgacggtacccactgccgctgcgcagcgggaaggaagctaagatcctacagcacttcggagacgggctctgccggatgctggacgagcggctgcagcggcaccgaacatcgggcggtgaccatgccccggactcaccatctggagagaacagtccagccccgcaggggcgacttgcggaagtccaggactcttccatgccagttcctgcccagcccaaagcgggaggctctggcagctactggccagctcggcactcaggagcccgagtgatactgctggtgctctaccgggagcacctgaatcctaatggtcaccacttcttaaccaaggaggagctgctgcagaggtgtgctcagaagtcccccagggtagcccctgggagtgccccaccctggccagccctccgctccctccttcacaggaacctggtcctcaggacacaccagccagccaggtactcattgaccccagagggcctggagctggcccagaagttggccgagtcagaaggcctgagcttgctgaatgtgggcatcgggcccaaggagccccctggggaggagacagcagtgccaggagcagcttcagcagagcttgccagtgaagcaggggtccagcagcagccactggagctgaggcctggagagtacagggtgctgttgtgtgtggacattggcgagacccgggggggcgggcacaggccggagctgctccgagagctacagcggctgcacgtgacccacacggtgcgcaagctgcacgttggagattttgtgtgggtggctcaggagaccaatcctagagacccagcaaaccctggggagttggtactggatcacattgtggagcgcaagcgactggatgacctttgcagcagcatcatcgacggccgcttccgggagcagaagttccgactgaagcgctgtggtctggagcgccgggtatacctggtggaagagcatggttccgtccacaacctcagccttcctgagagcacactgctgcaggctgtcaccaacactcaggtcattgatggcttttttgtgaagcgcacagcagacattaaggagtcagccgcctacctggccctcttgactcggggcctgcagagactctaccagggccacaccctacgcagccgcccctggggaacccctgggaaccctgaatcaggggccatgacctctccaaaccctctctgctcactcctcaccttcagtgacttcaacgcaggagccatcaagaataaggcccagtcggtgcgagaagtgtttgcccggcagctgatgcaggtgcgcggagtgagtggggagaaggcagcagccctggtggatcgatacagcacccctgccagcctcctggccgcctatgatgcctgtgccacccccaaggaacaagagacactgctgagcaccattaagtgtgggcgtctacagaggaatctggggcctgctctgagcaggaccttatcccagctctactgcagctacggccccttgacctgagcttatgccgtgaaacagcccccagcccccgtctgtcccccaacccaggctagccagccttttaacaacatcttttggggtacaattagaatctaagtgtttgcagccatatgtgtcatgtagaagatgcctagccctggggaccttgtgaaatacgcaggaaccagggataccatctggtccagtggtttttaaacaaagctgcttagcacctggaattccctggtcagggagatggagtcagtggggcattgcagcttggaatctattttatgtcaccagttggtcctcatcaaataaaatttccttaggagtgcagagggctcattgggaaaataaaaataataaaaataaataaaacttcctaaaagaaaagattgaaacccaaaaaaaaaaaaaaaaaaaaaaaaa

SEQ ID NO: 11 - protein, 463 aa, homo sapiens
MATVTATTKVPEIRDVTRIERIGAHSHIRGLGLDDALEPRQASQGMVGQLAARRAAGVVLEMIREGKIAGRAVLIAGQPGTGKTAIAMGMAQALGPDTPFTAIAGSEIFSLEMSKTEALTQAFRRSIGVRIKEETEIIEGEVVEIQIDRPATGTGSKVGKLTLKTTEMETIYDLGTKMIESLTKDKVQAGDVITIDKATGKISKLGRSFTRARDYDAMGSQTKFVQCPDGELQKRKEVVHTVSLHEIDVINSRTQGFLALFSGDTGEIKSEVREQINAKVAEWREEGKAEIIPGVLFIDEVHMLDIESFSFLNRALESDMAPVLIMATNRGITRIRGTSYQSPHGIPIDLLDRLLIVSTTPYSEKDTKQILRIRCEEEDVEMSEDAYTVLTRIGLETSLRYAIQLITAASLVCRKRKGTEVQVDDIKRVYSLFLDESRSTQYMKEYQDAFLFNELKGETMDTS

SEQ ID NO: 12 - DNA, 1488 bp, homo sapiens
gttggtgagcatcatggcaaccgttacagccacaaccaaagtcccggagatccgtgatgtaacaaggattgagcgaatcggtgcccactcccacatccggggactggggctggacgatgccttggagcctcggcaggcttcgcaaggcatggtgggtcagctggcggcacggcgggcggctggcgtggtgctggagatgatccgggaagggaagattgccggtcgggcagtccttattgctggccagccgggcacggggaagacggccatcgccatgggcatggcgcaggccctgggccctgacacgccattcacagccatcgccggcagtgaaatcttctccctggagatgagcaagaccgaggcgctgacgcaggccttccggcggtccatcggcgttcgcatcaaggaggagacggagatcatcgaaggggaggtggtggagatccagattgatcgaccagcaacagggacgggctccaaggtgggcaaactgaccctcaagaccacagagatggagaccatctacgacctgggcaccaagatgattgagtccctgaccaaggacaaggtccaggccggggacgtgatcaccatcgacaaggcgacgggcaagatctccaagctgggccgctccttcacacgcgcccgcgactacgacgctatgggctcccagaccaagttcgtgcagtgcccagatggggagctccagaaacgcaaggaggtggtgcacaccgtgtccctgcacgagatcgacgtcatcaactctcgcacccagggcttcctggcgctcttctcaggtgacacaggggagatcaagtcagaagtccgtgagcagatcaatgccaaggtggctgagtggcgcgaggagggcaaggcggagatcatccctggagtgctgttcatcgacgaggtccacatgctggacatcgagagcttctccttcctcaaccgggccctggagagtgacatggcgcctgtcctgatcatggccaccaaccgtggcatcacgcgaatccggggcaccagctaccagagccctcacggcatccccatagacctgctggaccggctgcttatcgtctccaccaccccctacagcgagaaagacacgaagcagatcctccgcatccggtgcgaggaagaagatgtggagatgagtgaggacgcctacacggtgctgacccgcatcgggctggagacgtcactgcgctacgccatccagctcatcacagctgccagcttggtgtgccggaaacgcaagggtacagaagtgcaggtggatgacatcaagcgggtctactcactcttcctggacgagtcccgctccacgcagtacatgaaggagtaccaggacgccttcctcttcaacgaactcaaaggcgagaccatggacacctcctgagttggatgtcatcccccgaccccaccctgttttccaccagagttctgacactgtgactctgtataaaatggttgggaagctgc

SEQ ID NO: 13 - protein, 345 aa, homo sapiens
MPARALLPRRMGHRTLASTPALWASIPCPRSELRLDLVLPSGQSFRWREQSPAHWSGVLADQVWTLTQTEEQLHCTVYRGDKSQASRPTPDELEAVRKYFQLDVTLAQLYHHWGSVDSHFQEVAQKFQGVRLLRQDPIECLFSFICSSNNNIARITGMVERLCQAFGPRLIQLDDVTYHGFPSLQALAGPEVEAHLRKLGLGYRARYVSASARAILEEQGGLAWLQQLRESSYEEAHKALCILPGVGTKVADCICLMALDKPQAVPVDVHMWHIAQRDYSWHPTTSQAKGPSPQTNKELGNFFRSLWGPYAGWAQAVLFSADLRQSRHAQEPPAKRRKGSKGPEG

SEQ ID NO: 14 - DNA, 2108 bp, homo sapiens
gaacccagaagaacacagctgtgcgcgcccacaggctctgggggcgggagaagataagtcgcaaggagggggcgggacctacacctcaggaaagccggagaattggggcacgaagcggggctttgatgacccgcaaagggcgaggcatgcaggaggtggaggaattaagtgaaacagggaaggttgttaaacagcaccgtgtgggcgaggccttaagggtcgtggtccttgtctgggcggggtctttgggcgtcgacgaggcctggttctgggtaggcggggctactacggggcggtgcctgctgtggaaatgcctgcccgcgcgcttctgcccaggcgcatggggcatcgtactctagcctccactcctgccctgtgggcctccatcccgtgccctcgctctgagctgcgcctggacctggttctgccttctggacaatctttccggtggagggagcaaagtcctgcacactggagtggtgtactagcggatcaagtatggacactgactcagactgaggagcagctccactgcactgtgtaccgaggagacaagagccaggctagcaggcccacaccagacgagctggaggccgtgcgcaagtacttccagctagatgttaccctggctcaactgtatcaccactggggttccgtggactcccacttccaagaggtggctcagaaattccaaggtgtgcgactgctgcgacaagaccccatcgaatgccttttctcttttatctgttcctccaacaacaacatcgcccgcatcactggcatggtggagcggctgtgccaggcttttggacctcggctcatccagcttgatgatgtcacctaccatggcttccccagcctgcaggccctggctgggccagaggtggaggctcatctcaggaagctgggcctgggctatcgtgcccgttacgtgagtgccagtgcccgagccatcctggaagaacagggcgggctagcctggctgcagcagctacgagagtcctcatatgaggaggcccacaaggccctctgcatcctgcctggagtgggcaccaaggtggctgactgcatctgcctgatggccctagacaagccccaggctgtgcccgtggatgtccatatgtggcacattgcccaacgtgactacagctggcaccctaccacgtcccaggcgaagggaccgagcccccagaccaacaaggaactgggaaactttttccggagcctgtggggaccttatgctggctgggcccaagcgggcctccttggcaatgcatttgatggccaccagcttctgcgtcctcttatcttctgccaggatcacctccgagaaggcccccctatcgggagaggggattcacaaggtgaagaactggaaccccagcttccctccagcctctcctccattccctatgggttctgtgaccactgctggaccaaggacgtggatgaccctcccctagtcactcatccatcccctggctccagagatggtcacatgacccaggcctggccagtcaaagtagtctctcccctggccacagtaattggtcatgtgatgcaagccagcttactagcactttgagaatgagtctcctgttgagctggtaggatgtaagcctggagctaatggcgatcatctttgccaccgcctggggagagcctgcttgggaatgaaattaacacaaaggaagtccaacctgagaaatggccaaatatatttcctgataacattatgtggccctctggatccagccatgcctgaggtctacccctgggcttttggattatgtgtacagttggttcatccctttttctgctaattcgagtcatggctaatttaacaccctttagaaccttaaagaaccatcagcatcacccgggaacttttttagaaatgcaaaatctctactgctttggatcctgggtcaaaaaaaagaaaaaaaaaagaaatgcaaaactttaggccctgccccagatttactaaatcaatctgcagtttaacaaaatcctcaggtgatttgtatgctcattgaactttaagaagcagtgttttagaacaggttcttaaaaaggaacaaataaactcatttaactaaagttaactg

SEQ ID NO: 15 - protein, 532 aa, homo sapiens
MNGVLIPHTPIAVDFWSLRRAGTARLFFLSHMHSDHTVGLSSTWARPLYCSPITAHLLHRHLQVSKQWIQALEVGESHVLPLDEIGQETMTVTLLDANHCPGSVMFLFEGYFGTILYTGDFRYTPSMLKEPALTLGKQIHTLYLDNTNCNPALVLPSRQEAAHQIVQLIRKHPQHNIKIGLYSLGKESLLEQLALEFQTWVVLSPRRLELVQLLGLADVFTVEEKAGRIHAVDHMEICHSNMLRWNQTHPTIAILPTSRKIHSSHPDIHVIPYSDHSSYSELRAFVAALKPCQVVPIVSRRPCGGFQDSLSPRISVPLIPDSVQQYMSSSSRKPSLLWLLERRLKRPRTQGVVFESPEESADQSQADRDSKKAKKEKLSPWPADLEKQPSHHPLRIKKQLFPDLYSKEWNKAVPFCESQKRVTMLTAPLGFSVHLRSTDEEFISQKTREEIGLGSPLVPMGDDDGGPEATGNQSAWMGHGSPLSHSSKGTPLLATEFRGLALKYLLTPVNFFQAGYSSRRFDQQVEKYHKPC

SEQ ID NO: 16 - DNA, 3664 bp, homo sapiens
cctttttctgcccactctggtaacttattgctctgctgggctctttcccttagggtctctggccctgttcttgccccagcatgacttttatcgggacgccgttgtggaagcctcacgcaggagccctgcccccgtggagaagatcccactggtgactccaaccctaccaccatgaatggggtcctgatcccccatacgcccatcgcagtggacttctggagcctgcgccgggctggcaccgcacgtctcttcttcttgtctcacatgcactcggaccacaccgtgggcctgtctagcacctgggcccggcccctctactgctccccaattacagcccacctcttgcatcgtcacctacaggtatctaagcaatggatccaagccctggaggttggtgagagccatgtattacccctagatgaaattggacaagagaccatgaccgtaaccctcctcgatgccaatcactgtcctggttctgtcatgtttctctttgaaggatattttggaaccatcctctacacaggtgattttcgatacacaccatccatgctaaaggagccagccctgacactggggaaacagatccatactttatacctagacaacaccaattgcaatccagccctggttcttccttcccgacaagaagctgcccaccagattgtccagctcattcgaaaacacccacaacataacataaagattggactctacagcctgggaaaggaatcactgctggagcagctggccctggagtttcagacctgggtggtattgagtcctcggcgcctggagttggtacagctactgggcctggcagatgtgttcacagtggaggagaaggctggccgcatccatgcagtagaccatatggagatctgccattccaacatgctgcgttggaaccagacccaccctacgattgctatccttcccacaagccgaaaaatccacagctcccaccctgatatccacgtcatcccttactctgaccattcctcttactccgagcttcgtgcctttgtcgcagcactgaagccttgccaggtggtgcccattgtaagtcggcggccctgtggaggctttcaggacagtctgagccccaggatctccgtgcccctgattccggactctgtacagcaatacatgagttcttcctctagaaaaccaagccttctctggctgttagaaaggaggctaaagaggccgagaacccaaggtgttgtgtttgaatcccctgaggaaagtgctgatcaatctcaagctgacagagactcaaagaaggccaagaaagagaaactttctccctggcctgcggaccttgaaaagcagccttcccaccatcctttgcggatcaagaagcagttgttcccagatctctatagcaaagaatggaacaaggcagtgcctttctgtgagtctcaaaagagggtgactatgttgacggccccactgggattttcagtgcacttaaggtctacagatgaggagtttatttctcaaaaaaccagggaggaaattggtttagggtcccccttggtacccatgggagatgatgatggaggtccagaagccacagggaatcagagtgcctggatgggccatggttctcccctgtcccacagcagcaagggcacccctcttctagctactgaattcaggggtctagcactcaaatatcttctgactccagtgaactttttccaggcagggtattcttccaggagatttgaccagcaagtggaaaaataccataaaccctgctgaagacaggagagtacagaatgacaacattgagcccacactgcagttttgaagatagtaactgatggctggtgggaaagagtttgtttttggggcctacttttctatctttacaagactcttatgggcccaccgtggagcagcacttcccaaaacttgttcactggggtcctcgtgcctatggaatccttctttttataactaagtttaagaaatactttttttataaaatctttggagtatgcgtgagcaaattaaaagttctttgaagtcctacagtaacttaatctgtttaaccttgtttaacccagtatttctcaaacttttgtgaacatgcaatcatcttatgtgggtacagaaagaggtaaagagtctgaatcaaaaaggaccaggttattgctgttgctgttttgtggtgtcatgagccattctccatgtccccttctccctcttctcagatcaaaatccctagggagttctatttttaaaattatgaactatggcgctgcatgcttcaatcctgaacgtcactgacttgctgtgaccatccaaataattttcctgtctctgcctctgggagggaacaggaagcgatgaagaggtcttggaacagtagtgaaaattctacctctatgtccttcatgaggatgtgcagtatcccagtatcactgggatccatgtggaacagagccagctggggggttgggcagctctctccaaggcagtacctagagcccagctgaacaacaaggctttgggtgtgaagggactccccagcctggagaccctatttggctgaaacagttacaaaatatcaaatgtgttgtcagatattcctccaattgttcacatagctgggatatttgttgctcccctcaccccttggattatgtagggagccagtgcacacagcctgtttgttttagtatccaaggaagagaccaaggagccagctggcgggaaggggtgggggtgtgcagtctgccctgtccttctgctcataacctgacaaaatgccaaactagtaagcaggatagctgataccacggctatgagggagtaggctctgagagggcacagacttgtggagctgggcgtctggatcaaaactgctttgggatggaacctcgagccctagcagtgaagaagattccatttcttgtccaggggatttaaaagagttttctgctttgagagagaaatagagagtttagaaagcaattgctcttgggaaagctatacacagctctgttttgtcaatgacctttgttgtaagtctcccaacgtcctattaggagccacagcaggtgaggcatttggtgcagcaggaaacatggggactgcctaggctcgaatctgtggcaccctgagcaattacttaaattgtggagcctagttcctcatctgtaagatggacttgagattcctacctctcatgattactatggagattgaataattggtaaaattctcctagctcagtgactgccacaggatgggtctttcagattttggttctctttagcttctggttcttgaaagaaattaatctgtatataacataagaaactttgaaagtcaaaaaaacaaaaaattttaattcctcgtagattaattgatttgctatcttttagtttttttttctatgcatgtagatgtattaaatatgtaaatgtttttcaaagttgaggtaatattgtatagaattttatagcctacattttaatttcttgatatcttaagcatttcacttattagatattgttcaaaaatgccatttttaatatttgtataataccctatcatgtgagtataccttaactaagccattcccatattcaacattttgtgtactgtttttctaattacatatattacaatgaaaaaaaaaaaaaaa

SEQ ID NO: 17 - protein, 1219 aa, homo sapiens
MPKIVLNGVTVDFPFQPYKCQQEYMTKVLECLQQKVNGILESPTGTGKTLCLLCTTLAWREHLRDGISARKIAERAQGELFPDRALSSWGNAAAAAGDPIACYTDIPKIIYASRTHSQLTQVINELRNTSYRPKVCVLGSREQLCIHPEVKKQESNHLQIHLCRKKVASRSCHFYNNVEEKSLEQELASPILDIEDLVKSGSKHRVCPYYLSRNLKQQADIIFMPYNYLLDAKSRRAHNIDLKGTVVIFDEAHNVEKMCEESASFDLTPHDLASGLDVIDQVLEEQTKAAQQGEPHPEFSADSPSPGLNMELEDIAKLKMILLRLEGAIDAVELPGDDSGVTKPGSYIFELFAEAQITFQTKGCILDSLDQIIQHLAGRAGVFTNTAGLQKLADIIQIVFSVDPSEGSPGSPAGLGALQSYKVHIHPDAGHRRTAQRSDAWSTTAARKRGKVLSYWCFSPGHSMHELVRQGVRSLILTSGTLAPVSSFALEMQIPFPVCLENPHIIDKHQIWVGVVPRGPDGAQLSSAFDRRFSEECLSSLGKALGNIARVVPYGLLIFFPSYPVMEKSLEFWRARDLARKMEALKPLFVEPRSKGSFSETISAYYARVAAPGSTGATFLAVCRGKASEGLDFSDTNGRGVIVTGLPYPPRMDPRVVLKMQFLDEMKGQGGAGGQFLSGQEWYRQQASRAVNQAIGRVIRHRQDYGAVFLCDHRFAFADARAQLPSWVRPHVRVYDNFGHVIRDVAQFFRVAERTMPAPAPRATAPSVRGEDAVSEAKSPGPFFSTRKAKSLDLHVPSLKQRSSGSPAAGDPESSLCVEYEQEPVPARQRPRGLLAALEHSEQRAGSPGEEQAHSCSTLSLLSEKRPAEEPRGGRKKIRLVSHPEEPVAGAQTDRAKLFMVAVKQELSQANFATFTQALQDYKGSDDFAALAACLGPLFAEDPKKHNLLQGFYQFVRPHHKQQFEEVCIQLTGRGCGYRPEHSIPRRQRAQPVLDPTGRTAPDPKLTVSTAAAQQLDPQEHLNQGRPHLSPRPPPTGDPGSQPQWGSGVPRAGKQGQHAVSAYLADARRALGSAGCSQLLAALTAYKQDDDLDKVLAVLAALTTAKPEDFPLLHRFSMFVRPHHKQRFSQTCTDLTGRPYPGMEPPGPQEERLAVPPVLTHRAPQPGPSRSEKTGKTQSKISSFLRQRPAGTVGAGGEDAGPSQSSGPPHGPAASEWGL

SEQ ID NO: 18 - DNA, 4462 bp, homo sapiens
ggaccggaagtggggggcggaagtgcagtgggctcagcgccgactgcgcgcctctgcccgcgaaaactctgagctggctgacagctggggacgggtggcggccctcgactggagtcggttgagttcctgagggaccccggttctggaaggttcgccgcggagacaagtgagcagtctgtgccatagggattctcgaagagaacagcgttgtgtcccagtgcacatgctcgcatcgcttaccaggagtgcccgagaccctaagatgttcggagtggttttttcgcacagacccgaatagcctgcccctcagccacgctctgtgcccttctgagaacaggctgatatgcccaagatagtcctgaatggtgtgaccgtagacttccctttccagccctacaaatgccaacaggagtacatgaccaaggtcctggaatgtctgcagcagaaggtgaatggcatcctggagagccctacgggtacagggaagacgctgtgcctgctgtgcaccacgctggcctggcgagaacacctccgagacggcatctctgcccgcaagattgccgagagggcgcaaggagagcttttcccggatcgggccttgtcatcctggggcaacgctgctgctgctgctggagaccccatagcttgctacacggacatcccaaagattatttacgcctccaggacccactcgcaactcacacaggtcatcaacgagcttcggaacacctcctaccggcctaaggtgtgtgtgctgggctcccgggagcagctgtgcatccatcctgaggtgaagaaacaagagagtaaccatctacagatccacttgtgccgtaagaaggtggcaagtcgctcctgtcatttctacaacaacgtagaagaaaaaagcctggagcaggagctggccagccccatcctggacattgaggacttggtcaagagcggaagcaagcacagggtgtgcccttactacctgtcccggaacctgaagcagcaagccgacatcatattcatgccgtacaattacttgttggatgccaagagccgcagagcacacaacattgacctgaaggggacagtcgtgatctttgacgaagctcacaacgtggagaagatgtgtgaagaatcggcatcctttgacctgactccccatgacctggcttcaggactggacgtcatagaccaggtgctggaggagcagaccaaggcagcgcagcagggtgagccccacccggagttcagcgcggactcccccagcccagggctgaacatggagctggaagacattgcaaagctgaagatgatcctgctgcgcctggagggggccatcgatgctgttgagctgcctggagacgacagcggtgtcaccaagccagggagctacatctttgagctgtttgctgaagcccagatcacgtttcagaccaagggctgcatcctggactcgctggaccagatcatccagcacctggcaggacgtgctggagtgttcaccaacacggccggactgcagaagctggcggacattatccagattgtgttcagtgtggacccctccgagggcagccctggttccccagcagggctgggggccttacagtcctataaggtgcacatccatcctgatgctggtcaccggaggacggctcagcggtctgatgcctggagcaccactgcagccagaaagcgagggaaggtgctgagctactggtgcttcagtcccggccacagcatgcacgagctggtccgccagggcgtccgctccctcatccttaccagcggcacgctggccccggtgtcctcctttgctctggagatgcagatccctttcccagtctgcctggagaacccacacatcatcgacaagcaccagatctgggtgggggtcgtccccagaggccccgatggagcccagttgagctccgcgtttgacagacggttttccgaggagtgcttatcctccctggggaaggctctgggcaacatcgcccgcgtggtgccctatgggctcctgatcttcttcccttcctatcctgtcatggagaagagcctggagttctggcgggcccgcgacttggccaggaagatggaggcgctgaagccgctgtttgtggagcccaggagcaaaggcagcttctccgagaccatcagtgcttactatgcaagggttgccgcccctgggtccaccggcgccaccttcctggcggtctgccggggcaaggccagcgaggggctggacttctcagacacgaatggccgtggtgtgattgtcacgggcctcccgtaccccccacgcatggacccccgggttgtcctcaagatgcagttcctggatgagatgaagggccagggtggggctgggggccagttcctctctgggcaggagtggtaccggcagcaggcgtccagggctgtgaaccaggccatcgggcgagtgatccggcaccgccaggactacggagctgtcttcctctgtgaccacaggttcgcctttgccgacgcaagagcccaactgccctcctgggtgcgtccccacgtcagggtgtatgacaactttggccatgtcatccgagacgtggcccagttcttccgtgttgccgagcgaactatgccagcgccggccccccgggctacagcacccagtgtgcgtggagaagatgctgtcagcgaggccaagtcgcctggccccttcttctccaccaggaaagctaagagtctggacctgcatgtccccagcctgaagcagaggtcctcagggtcaccagctgccggggaccccgagagtagcctgtgtgtggagtatgagcaggagccagttcctgcccggcagaggcccagggggctgctggccgccctggagcacagcgaacagcgggcggggagccctggcgaggagcaggcccacagctgctccaccctgtccctcctgtctgagaagaggccggcagaagaaccgcgaggagggaggaagaagatccggctggtcagccacccggaggagcccgtggctggtgcacagacggacagggccaagctcttcatggtggccgtgaagcaggagttgagccaagccaactttgccaccttcacccaggccctgcaggactacaagggttccgatgacttcgccgccctggccgcctgtctcggccccctctttgctgaggaccccaagaagcacaacctgctccaaggcttctaccagtttgtgcggccccaccataagcagcagtttgaggaggtctgtatccagctgacaggacgaggctgtggctatcggcctgagcacagcattccccgaaggcagcgggcacagccggtcctggaccccactggaagaacggcgccggatcccaagctgaccgtgtccacggctgcagcccagcagctggacccccaagagcacctgaaccagggcaggccccacctgtcgcccaggccacccccaacaggagaccctggcagccaaccacagtgggggtctggagtgcccagagcagggaagcagggccagcacgccgtgagcgcctacctggctgatgcccgcagggccctggggtccgcgggctgtagccaactcttggcagcgctgacagcctataagcaagacgacgacctcgacaaggtgctggctgtgttggccgccctgaccactgcaaagccagaggacttccccctgctgcacaggttcagcatgtttgtgcgtccacaccacaagcagcgcttctcacagacgtgcacagacctgaccggccggccctacccgggcatggagccaccgggaccccaggaggagaggcttgccgtgcctcctgtgcttacccacagggctccccaaccaggcccctcacggtccgagaagaccgggaagacccagagcaagatctcgtccttccttagacagaggccagcagggactgtgggggcgggcggtgaggatgcaggtcccagccagtcctcaggacctccccacgggcctgcagcatctgagtggggcctctaggatgtgcccagcctgccacaccgcctccaggaagcagagcgtcatgcaggtcttctggccagagccccagtgagtgcccacggaggcccccagcacacccaacgtggcttgatcacctgcctgtccagctctggtgggccaagaacccacccaacagaataggccagcccatgccagccggcttggcccgctgcaggcctcaggcaggcggggcccatggttggtccctgcggtgggaccggatctgggcctgcctctgagaagccctgagctaccttggggtctggggtgggtttctgggaaagtgcttccccagaacttccctggctcctggcctgtgagtggtgccacaggggcaccccagctgagcccctcaccgggaaggaggagacccccgtgggcacgtgtccacttttaatcaggggacagggctctctaataaagctgctggcagtgccc

SEQ ID NO: 19 - protein, 1367 aa, homo sapiens
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDPEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYENFIHLIIALPVAVLLIVGGLVIMLYVFHRKRNNSRLGNGVLYASVNPEYFSAADVYVPDEWEVAREKITMSRELGQGSFGMVYEGVAKGVVKDEPETRVAIKTVNEAASMRERIEFLNEASVMKEFNCHHVVRLLGVVSQGQPTLVIMELMTRGDLKSYLRSLRPEMENNPVLAPPSLSKMIQMAGEIADGMAYLNANKFVHRDLAARNCMVAEDFTVKIGDFGMTRDIYETDYYRKGGKGLLPVRWMSPESLKDGVFTTYSDVWSFGVVLWEIATLAEQPYQGLSNEQVLRFVMEGGLLDKPDNCPDMLFELMRMCWQYNPKMRPSFLEIISSIKEEMEPGFREVSFYYSEENKLPEPEELDLEPENMESVPLDPSASSSSLPLPDRHSGHKAENGPGPGVLVLRASFDERQPYAHMNGGRKNERALPLPQSSTC

SEQ ID NO: 20 - DNA, 4989 bp, homo sapiens
ttttttttttttttgagaaagggaatttcatcccaaataaaaggaatgaagtctggctccggaggagggtccccgacctcgctgtgggggctcctgtttctctccgccgcgctctcgctctggccgacgagtggagaaatctgcgggccaggcatcgacatccgcaacgactatcagcagctgaagcgcctggagaactgcacggtgatcgagggctacctccacatcctgctcatctccaaggccgaggactaccgcagctaccgcttccccaagctcacggtcattaccgagtacttgctgctgttccgagtggctggcctcgagagcctcggagacctcttccccaacctcacggtcatccgcggctggaaactcttctacaactacgccctggtcatcttcgagatgaccaatctcaaggatattgggctttacaacctgaggaacattactcggggggccatcaggattgagaaaaatgctgacctctgttacctctccactgtggactggtccctgatcctggatgcggtgtccaataactacattgtggggaataagcccccaaaggaatgtggggacctgtgtccagggaccatggaggagaagccgatgtgtgagaagaccaccatcaacaatgagtacaactaccgctgctggaccacaaaccgctgccagaaaatgtgcccaagcacgtgtgggaagcgggcgtgcaccgagaacaatgagtgctgccaccccgagtgcctgggcagctgcagcgcgcctgacaacgacacggcctgtgtagcttgccgccactactactatgccggtgtctgtgtgcctgcctgcccgcccaacacctacaggtttgagggctggcgctgtgtggaccgtgacttctgcgccaacatcctcagcgccgagagcagcgactccgaggggtttgtgatccacgacggcgagtgcatgcaggagtgcccctcgggcttcatccgcaacggcagccagagcatgtactgcatcccttgtgaaggtccttgcccgaaggtctgtgaggaagaaaagaaaacaaagaccattgattctgttacttctgctcagatgctccaaggatgcaccatcttcaagggcaatttgctcattaacatccgacgggggaataacattgcttcagagctggagaacttcatggggctcatcgaggtggtgacgggctacgtgaagatccgccattctcatgccttggtctccttgtccttcctaaaaaaccttcgcctcatcctaggagaggagcagctagaagggaattactccttctacgtcctcgacaaccagaacttgcagcaactgtgggactgggaccaccgcaacctgaccatcaaagcagggaaaatgtactttgctttcaatcccaaattatgtgtttccgaaatttaccgcatggaggaagtgacggggactaaagggcgccaaagcaaaggggacataaacaccaggaacaacggggagagagcctcctgtgaaagtgacgtcctgcatttcacctccaccaccacgtcgaagaatcgcatcatcataacctggcaccggtaccggccccctgactacagggatctcatcagcttcaccgtttactacaaggaagcaccctttaagaatgtcacagagtatgatgggcaggatgcctgcggctccaacagctggaacatggtggacgtggacctcccgcccaacaaggacgtggagcccggcatcttactacatgggctgaagccctggactcagtacgccgtttacgtcaaggctgtgaccctcaccatggtggagaacgaccatatccgtggggccaagagtgagatcttgtacattcgcaccaatgcttcagttccttccattcccttggacgttctttcagcatcgaactcctcttctcagttaatcgtgaagtggaaccctccctctctgcccaacggcaacctgagttactacattgtgcgctggcagcggcagcctcaggacggctacctttaccggcacaattactgctccaaagacaaaatccccatcaggaagtatgccgacggcaccatcgacattgaggaggtcacagagaaccccaagactgaggtgtgtggtggggagaaagggccttgctgcgcctgccccaaaactgaagccgagaagcaggccgagaaggaggaggctgaataccgcaaagtctttgagaatttcctgcacaactccatcttcgtgcccagacctgaaaggaagcggagagatgtcatgcaagtggccaacaccaccatgtccagccgaagcaggaacaccacggccgcagacacctacaacatcaccgacccggaagagctggagacagagtaccctttctttgagagcagagtggataacaaggagagaactgtcatttctaaccttcggcctttcacattgtaccgcatcgatatccacagctgcaaccacgaggctgagaagctgggctgcagcgcctccaacttcgtctttgcaaggactatgcccgcagaaggagcagatgacattcctgggccagtgacctgggagccaaggcctgaaaactccatctttttaaagtggccggaacctgagaatcccaatggattgattctaatgtatgaaataaaatacggatcacaagttgaggatcagcgagaatgtgtgtccagacaggaatacaggaagtatggaggggccaagctaaaccggctaaacccggggaactacacagcccggattcaggccacatctctctctgggaatgggtcgtggacagatcctgtgttcttctatgtccaggccaaaacaggatatgaaaacttcatccatctgatcatcgctctgcccgtcgctgtcctgttgatcgtgggagggttggtgattatgctgtacgtcttccatagaaagagaaataacagcaggctggggaatggagtgctgtatgcctctgtgaacccggagtacttcagcgctgctgatgtgtacgttcctgatgagtgggaggtggctcgggagaagatcaccatgagccgggaacttgggcaggggtcgtttgggatggtctatgaaggagttgccaagggtgtggtgaaagatgaacctgaaaccagagtggccattaaaacagtgaacgaggccgcaagcatgcgtgagaggattgagtttctcaacgaagcttctgtgatgaaggagttcaattgtcaccatgtggtgcgattgctgggtgtggtgtcccaaggccagccaacactggtcatcatggaactgatgacacggggcgatctcaaaagttatctccggtctctgaggccagaaatggagaataatccagtcctagcacctccaagcctgagcaagatgattcagatggccggagagattgcagacggcatggcatacctcaacgccaataagttcgtccacagagaccttgctgcccggaattgcatggtagccgaagatttcacagtcaaaatcggagattttggtatgacgcgagatatctatgagacagactattaccggaaaggaggcaaagggctgctgcccgtgcgctggatgtctcctgagtccctcaaggatggagtcttcaccacttactcggacgtctggtccttcggggtcgtcctctgggagatcgccacactggccgagcagccctaccagggcttgtccaacgagcaagtccttcgcttcgtcatggagggcggccttctggacaagccagacaactgtcctgacatgctgtttgaactgatgcgcatgtgctggcagtataaccccaagatgaggccttccttcctggagatcatcagcagcatcaaagaggagatggagcctggcttccgggaggtctccttctactacagcgaggagaacaagctgcccgagccggaggagctggacctggagccagagaacatggagagcgtccccctggacccctcggcctcctcgtcctccctgccactgcccgacagacactcaggacacaaggccgagaacggccccggccctggggtgctggtcctccgcgccagcttcgacgagagacagccttacgcccacatgaacgggggccgcaagaacgagcgggccttgccgctgccccagtcttcgacctgctgatccttggatcctgaatctgtgcaaacagtaacgtgtgcgcacgcgcagcggggtggggggggagagagagttttaacaatccattcacaagcctcctgtacctcagtggatcttcagttctgcccttgctgcccgcgggagacagcttctctgcagtaaaacacatttgggatgttccttttttcaatatgcaagcagctttttattccctgcccaaacccttaactgacatgggcctttaagaaccttaatgacaacacttaatagcaacagagcacttgagaaccagtctcctcactctgtccctgtccttccctgttctccctttctctctcctctctgcttcataacggaaaaataattgccacaagtccagctgggaagccctttttatcagtttgaggaagtggctgtccctgtggccccatccaaccactgtacacacccgcctgacaccgtgggtcattacaaaaaaacacgtggagatggaaatttttacctttatctttcacctttctagggacatgaaatttacaaagggccatcgttcatccaaggctgttaccattttaacgctgcctaattttgccaaaatcctgaactttctccctcatcggcccggcgctgattcctcgtgtccggaggcatgggtgagcatggcagctggttgctccatttgagagacacgctggcgacacactccgtccatccgactgcccctgctgtgctgctcaaggccacaggcacacaggtctcattgcttctgactagattattatttgggggaactggacacaataggtctttctctcagtgaaggtggggagaagctgaaccggc

(19) United States
(12) Patent Application Publication
(10) Pub. No.: US 2009/0221679 A1
(43) Pub. Date: Sep. 3, 2009

(54) Novel HIV Targets

(76) Inventors: Amy Espeseth, Chalfont, PA (US); Daria J. Hazuda, Doylestown, PA (US); Min Xu, Ambler, PA (US)

Correspondence Address: MERCK AND CO., INC, P O BOX 2000, RAHWAY, NJ, 07065-0907, US

(21) Appl. No.: 11/990,174

(22) PCT Filed: Aug. 8, 2006
(86) PCT No.: PCT/US06/30856

§ 371 Date: Feb. 8, 2008

Related U.S. Application Data
(60) Provisional application No. 60/707,012, filed on Aug. 10, 2005.
(60) Provisional application No. 60/708,738, filed on Aug. 16, 2005.

(51) Int. Cl. A61K 31/7088; C40B 30/04; C12Q 1/70

(52) U.S. Cl. 514/44 R; 506/9; 435/5

(57) ABSTRACT

Using a method to measure the effect of downregulation of certain cellular proteins on HIV integration, host proteins implicated in HIV infection were identified. The identified proteins and encoding nucleic acids provide targets for inhibiting HIV infection and for evaluating the ability of compounds to inhibit HIV infection. Compounds inhibiting HIV infection include compounds targeting identified proteins and compounds targeting nucleic acids encoding the proteins.

### BACKGROUND OF THE INVENTION

The references cited in the present application are not admitted to be prior art to the claimed invention.

After the Human Immunodeficiency Virus (HIV) integrates into the host genome, a gap remains between the integrated viral DNA and the host chromosome. Because HIV integrase is incapable of repairing the gap, the damage has long been assumed to be repaired by host DNA repair factors. Although it has been possible to model the enzymatic steps involved in repairing HIV integration-induced lesions in DNA in vitro (e.g., Yoder & Bushman, 2000), host factors that might be necessary for HIV transduction remain to be conclusively identified. A number of DNA repair-associated proteins have been linked to retroviral transduction as it is known that host DNA repair pathways are required to complete the process of retroviral integration (Kilzer, et al., 2003; Daniel, et al., 2004; Parissi, et al., 2003; Mulder et al., 2002). This indicates that such host cellular factors may be potential targets for antiviral therapy.

Past drug discovery programs for HIV have largely targeted viral enzymes, including reverse transcriptase, protease, and integrase. Compounds targeting these enzymes have become the standard treatment for HIV infection. Although anti-retroviral therapy successfully suppresses viral replication, the existence of latent viral reservoirs coupled with the poor fidelity of HIV reverse transcriptase often leads to the emergence of resistance. Because the pharmacological targeting of required host factors may slow or prevent viral resistance, the identification of novel host factors as targets for HIV therapy represents a significant advance for the field of HIV therapeutics.

Thus, there is an unmet need to identify novel targets for the treatment of HIV infection, which might include host cellular factors.

### SUMMARY OF THE INVENTION

A set of genes have been identified by siRNA screening as being essential for HIV infection. Knockdown of expression of these genes using siRNA decreases HIV transduction of P4/R5 HeLa cells in a single cycle HIV infectivity assay. The identified genes and proteins encoded thereby provide targets for inhibiting HIV infection and for evaluating the ability of compounds to inhibit HIV infection, which might include both compounds targeting the nucleic acids encoding the proteins identified and those targeting the proteins themselves.

Thus, in one embodiment of the present invention there is described a method of identifying a host cell factor involved in HIV infection using a siRNA library. A “library” contains a collection of different siRNAs screened as part of an experiment. The experimental results are obtained at about the same time or over a limited time period. In different embodiments, the limited time period is within about a week or within about a day. Preferably, the members of the library are tested at the same time. Reference to the library comprising a certain number of siRNA different host cell factors indicates that at least the indicated number of different siRNA are used. siRNA methods and compositions are set forth in references such as WO2005042708 and WO2005018534, the disclosures of which are incorporated herein by reference.

The method of identifying a host cell factor involved in HIV infection comprises the step of measuring the ability of a siRNA library targeting different host cell factors to inhibit HIV infection, wherein measuring the ability of a siRNA library to inhibit HIV infection further comprises: transfecting human cells with the siRNA library targeting different cell factors; infecting the transfected cells with HIV; and assaying for viral infection to determine whether siRNA-mediated downregulation of host cell factors inhibits HIV infection. More particularly, the siRNA library may comprise at least 244 different siRNA's targeting a different host cellular protein not previously associated with HIV infection. Additionally, the host cellular proteins may be one or more components of a DNA repair pathway.

In another embodiment of the invention there is provided isolated host cellular proteins involved in HIV infection selected from the group consisting of: post-meiotic segregation increased 2-like 1 (PMS2L1); excision repair cross-complementing rodent repair deficiency, complementation group 3 (ERCC3); DNA polymerase iota (POLI); transition protein 1 (TNP1); DNA polymerase lambda (POLL); centromere protein F (CENPF); MutS homolog 6 (MSH6); Nei-like 2 (NEIL2); B-cell translocation gene (BTG) family, member 2 (BTG2); damage-specific DNA binding protein 2 (DDB2); DNA cross-link repair 1B (DCLRE1b); regulator of telomere elongation helicase 1 (RTEL1); RAD51 homolog C (RAD51C); DNA polymerase epsilon (POLE); structural maintenance of chromosomes 6-like 1 (SMC6L1); AP endonuclease class 1 (APEX1); TATA box binding protein-associated factor, RNA polymerase II, (TAF2); 8-oxoguanine DNA glycosylase (OGG1); RuvB-like 2 (RUVBL2); RecQ protein-like 4 (RECQL4); topoisomerase (DNA) II alpha (TOP2A); Excision repair cross-complementing rodent repair deficiency, complementation group 3 (ERCC3); Replication protein A2 (RPA2); High mobility group (nonhistone chromosomal) protein 4-like (HMG4L); Retinoblastoma binding protein 8 (RBBP8); MutL homolog 1 (MLH1); MUS81 endonuclease homolog (MUS81); MutS homolog 4 (MSH4); Insulin-like growth factor 1 receptor (IGF1R); RAD23 homolog B (RAD23B); Ankyrin repeat domain 17 (ANKRD17); Nth endonuclease III-like 1 (NTHL1); DNA polymerase eta (POLH); WD repeat domain 33 (WDR33); DNA cross-link repair 1A (DCLRE1A), and Postmeiotic segregation increased 1 (PMS1), or a protein substantially similar to the target protein and homologs.

“Substantially similar” is defined as a sequence identity of at least 95% to the target protein. Nucleic acid and protein substantially similar to a particular identified sequence provide sequences with a small number of changes to the particular identified sequence. Substantially similar sequences include sequences containing one or more naturally occurring polymorphisms or changes that are artificially produced. A substantially similar protein sequence is at least 95% identical to a reference sequence. The substantially similar protein sequence should also not have significantly less activity than the reference sequence. In different embodiments, the substantially similar protein sequence differs from the reference sequence by 0, 1, 2, 3, 4, 5, 6, 7, 8, 9, 10, 11, 12, 13, 14, 15, 16, 17, 18, 19, or 20 amino acid alterations. Each amino acid alteration is independently an addition, deletion or substitution. Preferred substantially similar sequences are naturally occurring variants. A substantially similar nucleic acid is at least 95% identical to a reference sequence. The substantially similar nucleic acid sequence should encode a protein that does not have significantly less activity than the protein encoded by the reference sequence. In different embodiments, the substantially similar nucleic acid sequence differs from the reference sequence by 0, 1, 2, 3, 4, 5, 6, 7, 8, 9, 10, 11, 12, 13, 14, 15, 16, 17, 18, 19, or 20 nucleotide alterations. Each nucleic acid alteration is independently an addition, deletion or substitution. Preferred substantially similar sequences are naturally occurring variants.

In yet another embodiment of the invention, there is provided an assay for identifying a compound as an HIV inhibitor comprising the steps of: identifying a compound that downregulates or otherwise inhibits the activity or expression of a target protein that is a component of a DNA repair pathway of a human cell; and determining the ability of said compound to inhibit HIV. Said assay may be more particularly characterized in that the target protein is either or a protein having a sequence identity with one or more members selected from the group consisting of: PMS2L1; ERCC3; POLI; TNP1; POLL; CENPF; MSH6; NEIL2; BTG2; DDB2; DCLRE1b; RTEL1; RAD51C; POLE; SMC6L1; APEX1; TAF2; OGG1; RUVBL2; RECQL4; TOP2A; RPA2; HMG4L; RBBP8; MLH1; MUS81; MSH4; IGF1R; RAD23B; ANKRD17; NTHL1; POLH; WDR33; DCLRE1A, and PMS1 and homologs.

In another embodiment of the invention there is provided a method of identifying a biological pathway involved in HIV infection comprising the steps of: identifying genes targeted by siRNA analysis of host cellular genes whose downregulation inhibits HIV infection; inputting those genes into a database; and identifying what pathway they map to.

In yet another embodiment of the present invention there is provided a method of screening for a compound which down-regulates the expression of one or more components of a DNA repair pathway of a human cell, thereby decreasing HIV infection, comprising the steps of: contacting the one or more components of a DNA repair pathway of a human cell with a noncircularized HIV DNA in the presence of a test compound; contacting the or more components of a DNA repair pathway of a human cell with a noncircularized HIV DNA in the absence of a test compound; and determining the effect of the test compound on HIV integration as measured by the amount of circularization. More particularly, the one or more components of a DNA repair pathway of a human cell may be a nucleic acid molecule encoding a polypeptide selected from the group consisting of: PMS2L1; ERCC3; POLI; TNP1; POLL; CENPF; MSH6; NEIL2; BTG2; DDB2; DCLRE1b; RTEL1; RAD51C; POLE; SMC6L1; APEX1; TAF2; OGG1; RUVBL2; RECQL4; TOP2A; RPA2; HMG4L; RBBP8; MLH1; MUS81; MSH4; IGF1R; RAD23B; ANKRD17; NTHL1; POLH; WDR33; DCLRE1A, and PMS1 and homologs thereof.

### BRIEF DESCRIPTION OF THE DRAWINGS

FIG. 1 provides the protein sequence (1A) (SEQ ID NO: 1) and encoding cDNA sequence (1B) (SEQ ID NO: 2) for novel target PMS2L1.

FIG. 2 provides the protein sequence (2A) (SEQ ID NO: 3) and encoding cDNA sequence (2B) (SEQ ID NO: 4) for novel target ERCC3.

FIG. 3 provides the protein sequence (3A) (SEQ ID NO: 5) and encoding cDNA sequence (3B) (SEQ ID NO: 6) for novel target APEX1.

FIG. 4 provides the protein sequence (4A) (SEQ NO: 7) and encoding cDNA sequence (4B) (SEQ ID NO: 8) for novel target POLI.

FIG. 5 provides the protein sequence (5A) (SEQ ID NO: 9) and encoding cDNA sequence (5B) (SEQ ID NO: 10) for novel target MUS81.

FIG. 6 provides the protein sequence (6A) (SEQ ID NO: 11) and encoding cDNA sequence (6B) (SEQ ID NO: 12) for novel target RUVBL2.

FIG. 7 provides the protein sequence (7A) (SEQ ID NO: 13) and encoding cDNA sequence (7B) (SEQ ID NO: 14) for novel target OGG1.

FIG. 8 provides the protein sequence (8A) (SEQ ID NO: 15) and encoding cDNA sequence (8B) (SEQ ID NO: 16) for novel target DCLRE1b.

FIG. 9 provides the protein sequence (9A) (SEQ ID NO: 17) and encoding cDNA sequence (9B) (SEQ ID NO: 18) for novel target RTEL1.

FIG. 10 provides the protein sequence (10A) (SEQ ID NO: 19) and encoding DNA sequence (10B) (SEQ ID NO: 20) for novel target IGFR1.

### DETAILED DESCRIPTION OF THE INVENTION

Novel host cell protein targets for inhibiting HIV infection have been identified. Such targets may prove useful not only for inhibiting HIV infection, but also for assessing the ability of compounds to inhibit HIV infection.

A library of siRNAs targeting genes involved in DNA repair was transfected into HeLa P4/R5 cells. P4/R5 is a cell line which stably expresses exogenous CD4, CCR5 and LTR-β-GAL. Twenty-four hours following siRNA transfection, the cells were infected with HIV. Forty-eight hours after infection, the cells were assayed for expression of the β-GAL reporter gene, as an indication that the virus had successfully integrated into the host genome and was producing sufficient quantities of the viral Tat protein to induce expression through the LTR (Joyce et al., 2002). siRNAs that blocked or reduced the expression of β-GAL were then examined in more detail.

Cells were transfected with a pool of three siRNAs targeting each gene at 50 nM final concentration. siRNAs targeting 242 genes with Gene Ontology annotations indicating an involvement in DNA repair were assayed in duplicate in both the presence and absence of an HIV integrase inhibitor. Transfections of siRNAs targeting cyclin T1 and CDK9 were included as positive controls for each transfection plate. Mock transfections and transfections of a non-silencing siRNA directed against luciferase were included as negative controls for each transfection plate. Two days after infection, the cells were lysed and β-GAL activity was assayed. A “hit” was defined as any siRNA pool that decreased β-galactosidase activity by more than 40% relative to controls, or that showed enhanced effects on HIV infection in the presence of EC50 concentrations of an integrase inhibitor. All of these siRNA pools were chosen for further analysis. siRNAs from each original pool of three siRNAs were assayed individually for their effect on HIV infection. If two out of the three siRNAs in the pool were effective inhibitors, the hit was considered to be confirmed.

Inhibiting HIV infection has implications for both research and for antiviral therapy. Research applications of the present invention include providing methods to screen for compounds which inhibit HIV infection. Therapeutic applications include using identified compounds to treat or inhibit HIV infection.

### EXAMPLES

Examples are provided below further illustrating different features of the present invention and illustrate useful embodiments for practicing the invention. Theses embodiments should be viewed as exemplary of the present invention rather than in any way limiting its scope.

### Example 1

### Identification of DNA Repair Genes Involved in HIV Infection

The procedure was performed as follows:

Day 1: Plate HeLa (P4/R5) cells at 2000 cells per well in 4×96-well plates.
Day 2: Transfect HeLa (P4/R5) cells with siRNA pools as follows:
1. siRNAs will be transfected at a final concentration of 50 nM using a transfection reagent, such as OLIGOFECTAMINE™ reagent (Invitrogen), at a final concentration of 0.5%. Positive and negative control siRNAs are included as follows:
CDK9 (positive control): GUGGUCAACUUGAUUGAGAdTdT
Cyclin T1 (positive control): purchased from Santa Cruz Biotechnology (Cat. No. sc-35144)
Luciferase (negative control): CGUACGCGGAAUACUUCGAdTdT
2. Dispense 66 μL of OptiMEM/well into a sterile 96-well plate, leaving the 12^{th} column empty.
3. Transfer 2 μL of siRNA (resuspended at 10 μM) from each well of the siRNA stock plate into the OptiMEM-containing plates such that the siRNA from well A3 of the mother plate is transferred into well A2 of the daughter plate (2 μL of the siRNA from each well is transferred into the corresponding plate into the same row position and the N−1 column position).
4. Mix by pipetting up and down.
5. In a tube, add 240 μL Oligofectamine, 1210 μL OptiMEM. Incubate 5 minutes at room temperature.
6. Dispense 12 μL of the Oligofectamine to each well and mix by pipetting up and down.

Incubate the plate at room temperature for 15 minutes.
7. Add 20 μL of the siRNA-Oligofectamine complex to each well of the HeLa (P4/R5) cells.
   Day 3: Transfected HeLa (P4/R5) cells were infected with HXB2 HIV in the presence and absence of an integrase inhibitor as follows:
1. Media was removed from the cells.
2. 80 μL fresh media was added to each well.
3. Integrase inhibitor was diluted to 20 nM in media. 40 μL of the 20 nM solution of integrase inhibitor was added to each well of two of the plates (the final concentration of integrase inhibitor was equal to the IC50 of the compound for inhibition of viral infection in this assay (Anthony et al., 2004)). 40 μL of the media without compound was added to the remaining two plates.
4. HXB2 HIV was diluted with media 100×. 40 μL of diluted HXB2 was added to each well.
5. Viral infection was allowed to proceed for 48 hours.
   Day 5: Beta-galactosidase activity, an indication of viral infection, was measured as follows:
1. Media was removed from the cells.
2. Cells were washed with 200 μL PBS per well.
3. 20 μL lysis buffer (such as buffer from the GALACTO-LIGHT PLUS™ assay system, Applied Biosystems) containing DTT was added to each well, and the plates were shaken for 10 minutes.
4. 80 μL of substrate was then added to each well and the plates were incubated at room temperature in the dark for 1 hour.
5. 100 μl of an enhancing solution was added to each well and the plates were read using a Dynex luminometer.

Data was analyzed in the following manner. Readings for each plate were normalized to the reading for the luciferase negative control and expressed as “percent of Luciferase Control”. Hits were considered to be those siRNA pools that suppressed beta-galactosidase activity by 40% or more, or those that showed 30% or greater inhibition of beta-galactosidase activity in the presence of IC50 levels of integrase inhibitor compared to the absence of compound treatment.

### Results:

The total number of inhibitory hits from the primary screen was 41, and included the following genes: SF3B3, PMS2L1, POLL, TNP1, POLL, CENPF, MSH6, NEIL2, SUPT3H, BTG2, DDB2, DCLRE1B, RAD51C, POLE, SMC6L1, APEX1, TAF2, OGG1, POLR2G, RUVBL2, RECQL4, TOP2A, ERCC3, RPA2, RRM2, HMG4L, RBBP8, MLH1, MUS81, MSH4, IGF1R, RAD23B, ANKRD17, NTHL1, POLH, WDR33, and DCLRE1A. An additional three genes were of interest because siRNAs targeting these genes appeared to enhance HIV infectivity. These genes were also considered to be hits: PMS1, HMGB2, XAB2.

Genes targeted by siRNAs that hit in the assay were evaluated further with respect to tissue distribution and which specific DNA repair pathways they represented. In addition, the siRNA hits were electronically counterscreened to assess whether they were toxic to HeLa cells in a viability-output screen. Additionally, the efficacy of the siRNA used in knocking down RNA or protein levels of the targeted gene was confirmed for ERCC3, MUS81, POL1, and RUVBL2 by testing mRNA levels with and without siRNA treatment, and for APEX1 and LIG3 by testing protein levels with and without siRNA treatment.

### Example 2

### Electronic Counterscreening of siRNA Hits

A siRNA screen was run in HeLa cells in which the cells were transfected with siRNAs and cell viability was assessed by Alamar Blue staining 72 h post-transfection. Thus, siRNAs that were toxic to HeLa cells in this assay may appear to hit in the infectivity screen simply due to cytotoxicity. For this reason, the siRNA hits from the HIV infection assay were examined for cytotoxic effects in the HeLa cytotoxicity assay.

### Results:

Analysis of the HeLa cytotoxicity data led to the elimination of six hits. The remaining hits of interest are: PMS2L1, RAD52, POLI, TNP1, POLL, CENPF, MSH6, NEIL2, BTG2, DDB2, DCLRE1B, C20orf41 (RTEL), ADPRT (PARP1), RAD51C, POLE, SMC6L1, APEX1, TAF2, OGG1, RUVBL2, RECQL4, TOP2A, ERCC3, RPA2, HMG4L, RBBP8, MLH1, MUS81, MSH4, IGF1R, XRCC4, RAD23B, ANKRD17, NTHL1, POLH, WDR33, DCLRE1A, and PMS1.

### Example 3

### Pathway Mapping Highlights the Base-Excision Repair Pathway

Genes targeted by siRNAs that hit in the HIV infection screen were analyzed using software that searches a database of gene ontology definitions and reports on gene ontology functions and pathways that are held in common by the query set. Excluding “DNA repair” as a definition, since this was the criteria used to define the gene set in the library, the following definitions were the top ten selections (displayed in Table 1):

TABLE 1

| | | | Overlap | Set Gene | |
|---|---|---|---|---|---|
| Similar Set | p-value | Expectation | gene count | count | Input identifiers |
| DNA | 0 | 0 | 14 | 110 | RUVBL2; POLI; POLL; MSH6; |
| recombination | | | | | MLH1; WDR33; RAD51C; |
| | | | | | RAD52; RPA2; XRCC4; MUS81 |
| DNA-dependent | 0 | 0 | 13 | 149 | POLI; ANKRD17; MSH6; |
| DNA replication | | | | | HMGB2; MLH1; PMS1; PMS2L1; |
| | | | | | POLE; POLH; RAD23B; RECQL4 |
| DNA-(apurinic or | 0.000000000009 | 0.000000032 | 5 | 10 | POLI; NEIL2; APEX1; |
| apyrimidinic site) | | | | | NTHL1; OGG1 |
| lyase activity | | | | | |
| Damaged DNA | 0.00000000016 | 0.000000055 | 15 | 74 | DDB2; ERCC3; SF3B3; ANKRD17; |
| binding | | | | | MSH4; OGG1; POLE; POLH; |
| | | | | | RAD23B; RAD51C; RPA2; |
| | | | | | XRCC4; SUPT3H |
| Base excision | 0.000000000017 | 0.000000057 | 8 | 35 | POLI; NEIL2; POLL; HMGB2; |
| repair | | | | | APEX1; OGG1; RPA2 |
| Nucleotide- | 0.0000000000021 | 0.000000071 | 11 | 51 | DDB2; ERCC3; POLL; MSH6; |
| excision repair | | | | | MLH1; POLR2G; XAB2; |
| | | | | | RAD23B; RPA2 |
| DNA replication | 0.0000000000025 | 0.000000084 | 16 | 242 | POLI; ANKRD17; POLL; MSH6; |
| | | | | | HMGB2; MSH4; PMS1; PMS2L1; |
| | | | | | POLE; POLH; RPA2; RRM2; |
| | | | | | TOP2A; RECQL4 |
| Transcription- | 0.00000000003 | 0.00000010695 | 6 | 16 | ERCC3; MSH6; MLH1; NTHL1; |
| coupled | | | | | POLR2G; XAB2 |
| nucleotide- | | | | | |
| excision repair | | | | | |
| Mismatch repair | 0.00000000003 | 0.00000010758 | 6 | 29 | ANKRD17; MSH6; MLH1; |
| | | | | | MSH4; PMS1, PMS2L1 |

After general functions, such as “DNA recombination”, “DNA-dependent DNA replication”, “DNA (apurinic or apyrimidinic site) lyase activity”, and “damaged DNA binding”, the highest ranked repair pathway was “base excision repair” or BER. Further analysis of the hits revealed that most of the hits mapped to the short patch repair pathway of BER, but some genes representing important BER functions did not hit in the screen. These include LIG3, MUTYH, POLB, and XRCC1. Additional siRNAs for these genes were tested for knockdown of HIV infection. Six individual siRNAs were tested for LIG3, MUTYH, POLB, and XRCC1. Of the six LIG3, MUTYH, and POLB siRNAs tested, three were capable of inhibiting HIV infection by 40% or more, confirming that these genes in the BER pathway are associated with HI V infection. Of the six XRCC1 siRNAs tested, two were capable of inhibiting HIV infection by 40% or more. Thus, the BER DNA repair pathway appears essential for HIV infection.

### Example 4

### Analysis of Tissue Distribution of Hits

siRNAs chosen for further analysis were examined for expression in cells infected by HIV or tissues that harbor the virus, including CD4+ T-lymphocytes, macrophage, lymph node and thymus using a previously generated Body Atlas, which contains data from microarray experiments carried out with many different tissues compared against a species-specific reference pool. Expression of all of the hits was examined in CD4+ T-lymphocytes, macrophage, lymph node and thymus.

### Results:

All of the genes had some expression in the cell types of interest, but some had higher expression levels in those tissues than others. The potential targets for HIV were grouped according to their tissue distribution, with high to moderate levels of expression in the tissues of interest being preferred. The ranking of targets proceeded as follows:
Tier 1 (high expression in CD4+ T lymphocytes, macrophage, lymph node, and thymus): PMS2 μl, MLH1, ERCC3, POLH, POLE, DCRLE1B, APEX1, POLI
Tier 2: (moderate to high expression in CD4+ T lymphocytes, macrophage, lymph node, and thymus): RBBP8, CENPF, TOP2A, DCRLE1A, TAF2, PMS1, SMC6L1, POLB, RAD51C, XRCC4, PARP1, DDB2, WDR33, RPA2
Tier 3: (moderate expression in CD4+ T lymphocytes, macrophage, lymph node, and thymus): XRCC1, OGG1, BTG2, HMG4L, RECQL4
Tier 4: (moderate to low expression in CD4+ T lymphocytes, macrophage, lymph node, and thymus): ANKRD17, RTEL1, NTHL1, POLL, MSH4, RUVBL2, LIG3, RAD23B, NEIL2, MUS81
Tier 5: (low expression in CD4+ T lymphocytes, macrophage, lymph node, and thymus): TNP1, IGF1R, MSH6, RAD52

The siRNAs in Example 1 were then re-assayed as individual siRNAs to guard against off-target activity arising from any one of the individual siRNAs present in the initial pool. Each of the individual siRNAs was tested for inhibition of HIV infection using the methodology described in Example 1. The hit was considered to be confirmed if a minimum of two out of the three siRNAs inhibited β-galactosidase activity by a minimum of 40% relative to the luciferase siRNA negative control.

After compiling the data from screening pools and individual siRNAs for effects of knockdown on HIV infection, followed by electronic counterscreening, pathway mapping, tissue distribution, and the potential for druggable domains, the siRNA hits were ranked and then prioritized as follows:
Tier 1: PMS2L1, PARP1, ERCC3, APEX1, POLI, RAD52, MUS81, RUVBL2
Tier 2: OGG1, IGFR1, RAD51C, DCLRE1B, DDB2, RTEL, POLL, MLH1, RECQL4, POLE
Tier 3: TNP1, LIG3, RBBP8, CENPF, POLB, BTG2, POLH, SMC6L1, RAD23B, XRCC4
Tier 4: WDR33, TAF2, NTHL1, MUTHY, MSH6, PMS1, RPA2, DCLRE1A, MSH4, ANKRD17, HMG4L, XRCC1, NEIL2, TOP2A

As indicated above, some of the genes identified in the screen have a published link to HIV, including PARP1 (Kameoka et al., 2004), XRCC4 (Daniel et al., J Virol. 78:8573, 2004), and RAD52 (Lau et al., 2004). Identification of the same genes through siRNA screening demonstrates that this screening method can effectively isolate genes with a known interaction with HIV. The remaining genes have no published link to HIV and represent truly novel targets for treatment of HIV infection.

### Example 5

### Novel Targets

Preferred genes identified by the screening methodology of the present invention include the following:

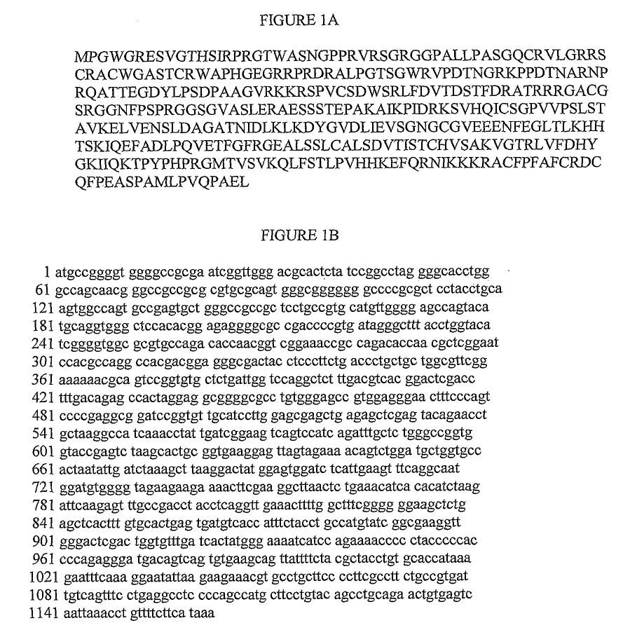

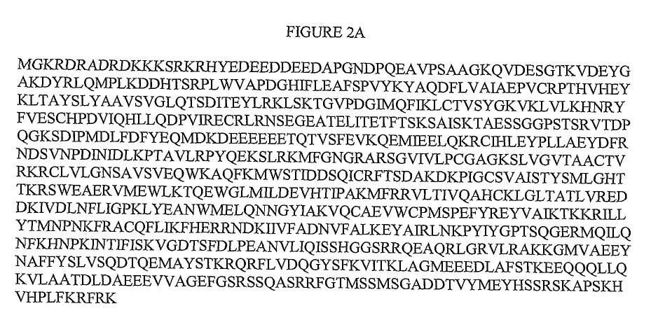

PMS2L1: (Postmeiotic segregation increased 2-like 1) which is a member of a family of proteins related to predicted DNA mismatch repair protein PMS2. The protein sequence and encoding cDNA sequence are provided in FIGS. 1A and B. PMS2L1 polymorphisms are shown in Table 2, derived from the NCBI single nucleotide polymorphism database. “Function” refers to the function of the nucleotide in each row. If the polymorphism corresponds to the sequence displayed as the standard reference sequence, it is designated as “contig reference”. If the polymorphism represents a nucleotide change that does not change the amino acid sequence, it is marked “synonymous”. A nucleotide change that changes the amino acid sequence, is designated as “nonsynonymous”.

TABLE 2
SNPs

| | | | | Amino |
|---|---|---|---|---|
| | dbSNP | Protein | Codon | acid |
| Function | allele | residue | position | position |
| synonymous | C | Ser [S] | 3 | 327 |
| contig reference | T | Ser [S] | 3 | 327 |
| nonsynonymous | G | Arg [R] | 2 | 320 |
| contig reference | A | His [H] | 2 | 320 |
| synonymous | T | Pro [P] | 3 | 319 |
| contig reference | C | Pro [P] | 3 | 319 |
| synonymous | A | Ser [S] | 3 | 297 |
| contig reference | G | Ser [S] | 3 | 297 |
| nonsynonymous | T | Leu [L] | 2 | 202 |
| contig reference | C | Pro [P] | 2 | 202 |
| nonsynonymous | T | Cys [C] | 3 | 56 |
| contig reference | G | Trp [W] | 3 | 56 |
| nonsynonymous | C | Thr [T] | 2 | 23 |
| contig reference | A | Asn [N] | 2 | 23 |

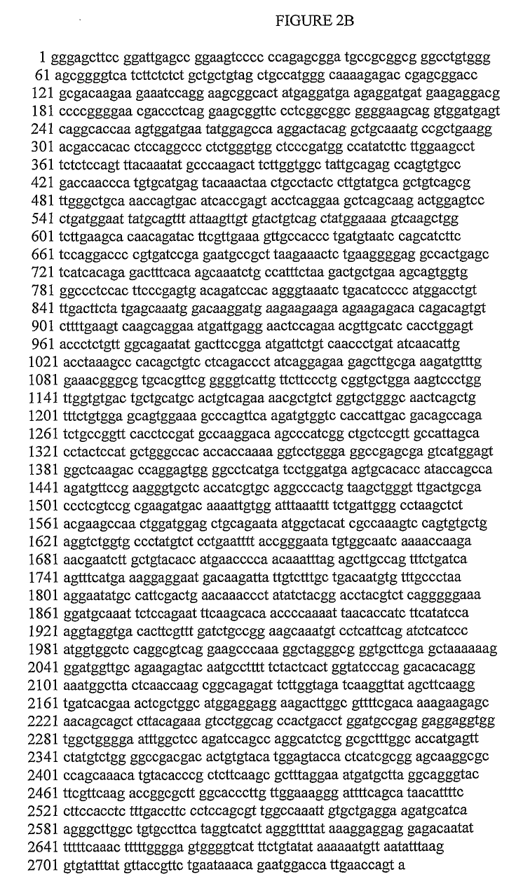

ERCC3: (Excision repair cross-complementing rodent repair deficiency (complementation group 3)) which is a DNA helicase involved in DNA repair and a member of the TFIIH transcriptional complex. The protein sequence and encoding cDNA sequence are provided in FIGS. 2A and B. ERCC3 polymorphisms are shown in Table 3:

TABLE 3
SNPs

| | dbSNP | Protein | Codon | Amino acid |
|---|---|---|---|---|
| Function | allele | residue | position | position |
| nonsynonymous | C | Pro [P] | 1 | 735 |
| contig reference | T | Ser [S] | 1 | 735 |
| nonsynonymous | T | Leu [L] | 2 | 704 |
| contig reference | C | Ser [S] | 2 | 704 |
| synonymous | T | Ile [I] | 3 | 580 |
| contig reference | C | Ile [I] | 3 | 580 |
| synonymous | A | Glu [E] | 3 | 495 |
| contig reference | G | Glu [E] | 3 | 495 |
| synonymous | T | Thr [T] | 3 | 445 |
| contig reference | C | Thr [T] | 3 | 445 |
| nonsynonymous | T | Cys [C] | 1 | 402 |
| contig reference | G | Gly [G] | 1 | 402 |
| synonymous | A | Gln [Q] | 3 | 373 |
| contig reference | G | Gln [Q] | 3 | 373 |
| synonymous | A | Glu [E] | 3 | 205 |
| contig reference | G | Glu [E] | 3 | 205 |
| contig reference | A | Lys [K] | 2 | 117 |

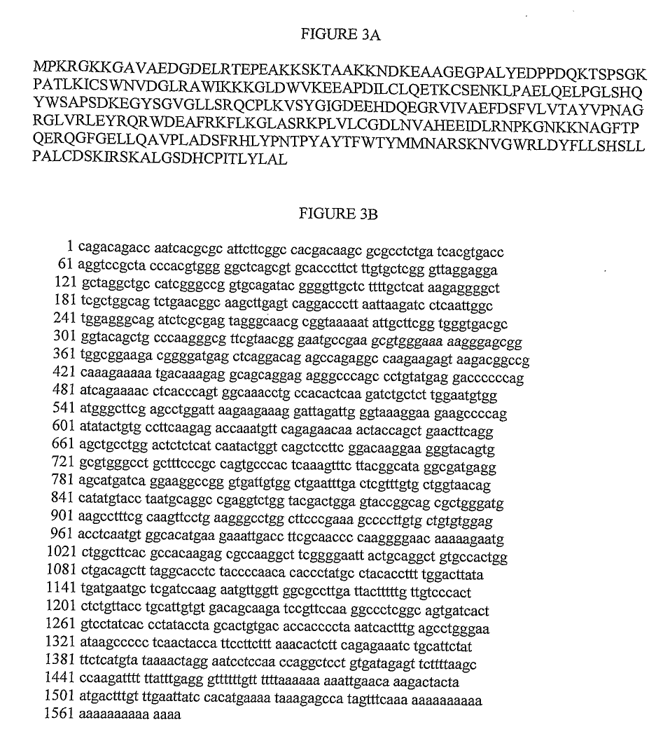

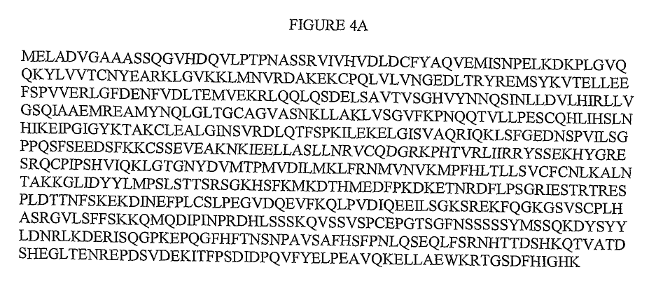

APEX1: (apurinic:apyrimidinic endonuclease I), which is a multifunctional DNA repair enzyme involved in the oxidative stress response. The protein sequence and encoding cDNA sequence are provided in FIGS. 3A and B. APEX1 polymorphisms are shown in Table 4:

TABLE 4
SNP

| | dbSNP | Protein | Codon | Amino acid |
|---|---|---|---|---|
| Function | allele | residue | position | position |
| nonsynonymous | C | His [H] | 3 | 51 |
| contig reference | G | Gln [Q] | 3 | 51 |
| nonsynonymous | G | Val [V] | 1 | 64 |
| contig reference | A | Ile [I] | 1 | 64 |
| nonsynonymous | G | Glu [E] | 3 | 148 |
| contig reference | T | Asp [D] | 3 | 148 |
| synonymous | C | Tyr [Y] | 3 | 269 |
| contig reference | T | Tyr [Y] | 3 | 269 |
| synonymous | C | Leu [L] | 1 | 286 |
| contig reference | T | Leu [L] | 1 | 286 |
| nonsynonymous | T | Ser [S] | 1 | 311 |
| contig reference | C | Pro [P] | 1 | 311 |
| nonsynonymous | T | Val [V] | 2 | 317 |
| contig reference | C | Ala [A] | 2 | 317 |

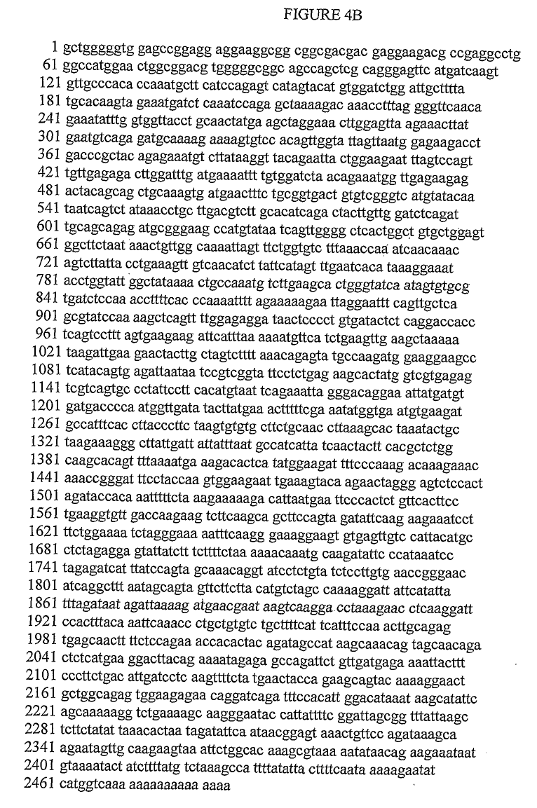

POLI: a low fidelity DNA polymerase and 5′-deoxyribose phosphate lyase that functions in translesion DNA replication and base excision DNA repair. The protein sequence and encoding cDNA sequence are provided in FIGS. 4A and B. POLI polymorphisms are shown in Table 5:

TABLE 5
SNPs

| | dbSNP | Protein | Codon | Amino acid |
|---|---|---|---|---|
| Function | allele | residue | position | position |
| synonymous | A | Ala [A] | 3 | 4 |
| contig reference | G | Ala [A] | 3 | 4 |
| nonsynonymous | G | Gly [G] | 1 | 71 |
| contig reference | A | Arg [R] | 1 | 71 |
| synonymous | C | Leu [L] | 1 | 191 |
| contig reference | T | Leu [L] | 1 | 191 |
| nonsynonymous | G | Met [M] | 3 | 236 |
| contig reference | A | Ile [I] | 3 | 236 |
| nonsynonymous | A | Lys [K] | 1 | 251 |
| contig reference | G | Glu [E] | 1 | 251 |
| nonsynonymous | A | Asn [N] | 1 | 349 |
| contig reference | T | Tyr [Y] | 1 | 349 |
| synonymous | G | Val [V] | 3 | 368 |
| contig reference | A | Val [V] | 3 | 368 |
| nonsynonymous | G | Arg [R] | 2 | 449 |
| contig reference | A | His [H] | 2 | 449 |
| nonsynonymous | C | Ser [S] | 2 | 507 |
| contig reference | T | Phe [F] | 2 | 507 |
| nonsynonymous | C | Arg [R] | 1 | 535 |
| contig reference | T | Cys [C] | 1 | 535 |
| nonsynonymous | A | Thr [T] | 1 | 706 |
| contig reference | G | Ala [A] | 1 | 706 |

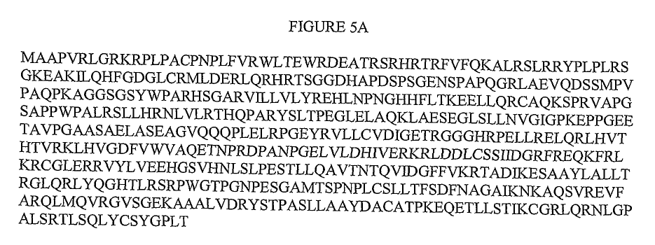

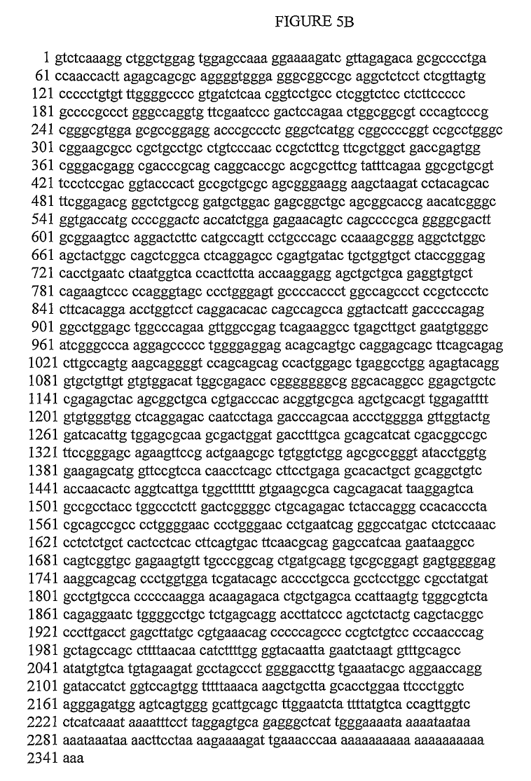

MUS81: MUS81 endonuclease is an endonuclease that cleaves Holliday junctions and it may be involved in the resolution of Holliday junctions formed during DNA replication responses to damage. FIG. 5 provides the protein sequence (5A) and encoding cDNA sequence (5B) for MUS81. MUS81 polymorphisms are shown in Table 6.

TABLE 6
SNP

| | dbSNP | Protein | Codon | Amino acid |
|---|---|---|---|---|
| Function | allele | residue | position | position |
| nonsynonymous | A | His [H] | 2 | 37 |
| contig reference | G | Arg [R] | 2 | 37 |
| synonymous | C | Ala [A] | 3 | 179 |
| contig reference | T | Ala [A] | 3 | 179 |
| nonsynonymous | C | Pro [P] | 2 | 180 |
| contig reference | G | Arg [R] | 2 | 180 |
| nonsynonymous | T | Phe [F] | 1 | 189 |
| contig reference | C | Leu [L] | 1 | 189 |
| synonymous | T | Ala [A] | 3 | 312 |
| contig reference | C | Ala [A] | 3 | 312 |
| synonymous | A | Arg [R] | 3 | 355 |
| contig reference | G | Arg [R] | 3 | 355 |
| synonymous | T | Thr [T] | 3 | 416 |
| contig reference | G | Thr [T] | 3 | 416 |
| nonsynonymous | C | His [H] | 3 | 481 |
| contig reference | G | Gln [Q] | 3 | 481 |

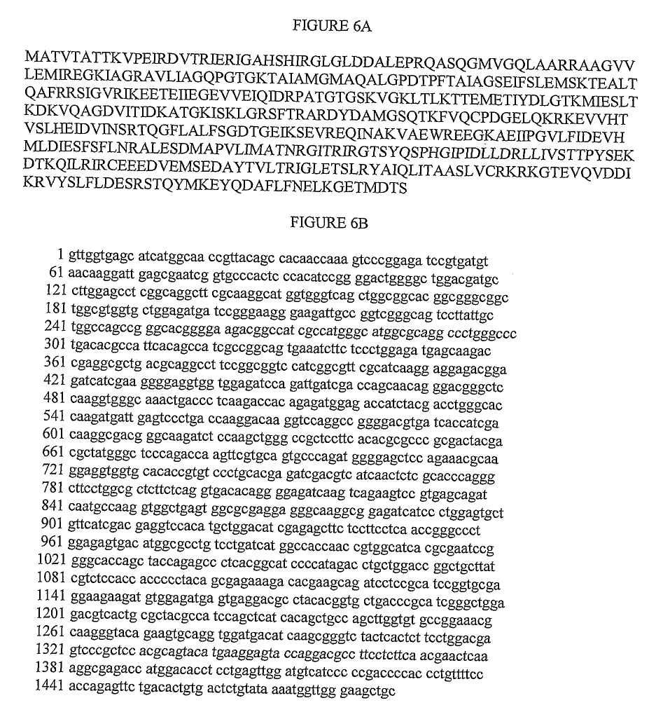

RUVBL2: (RUVB (E. coli)-like 2), which is a single-stranded DNA-stimulated ATPase and ATP-dependent DNA helicase. It is predicted to function in processes involved in DNA metabolism. FIG. 6 provides the protein sequence (6A) and encoding cDNA sequence (6B) for novel target POLI. RUVBL2 polymorphisms are shown in Table 7.

TABLE 7

| | dbSNP | Protein | Codon | Amino acid |
|---|---|---|---|---|
| Function | allele | residue | position | position |
| synonymous | C | Ala [A] | 3 | 56 |
| contig reference | T | Ala [A] | 3 | 56 |
| nonsynonynymous | A | Gln [Q] | 2 | 79 |
| contig reference | C | Pro [P] | 2 | 79 |
| synonymous | T | Leu [L] | 1 | 205 |
| contig reference | C | Leu [L] | 1 | 205 |
| synonymous | T | Leu [L] | 3 | 205 |
| contig reference | G | Leu [L] | 3 | 205 |
| synonymous | A | Lys [K] | 3 | 269 |
| contig reference | G | Lys [K] | 3 | 269 |

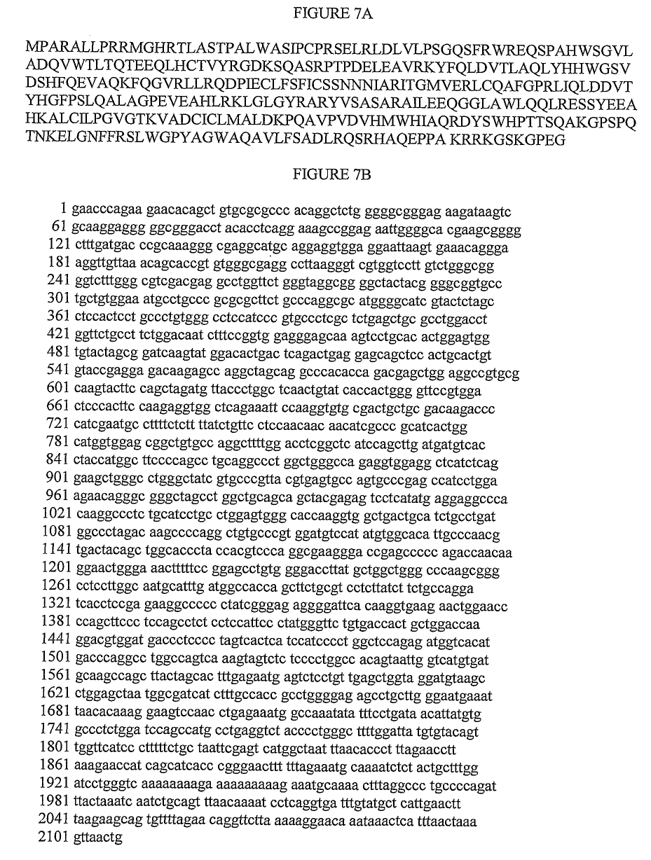

OGG1: (8-oxoguanine DNA glycosylase 1), which is a nuclear and mitochondrial base excision repair DNA enzyme that also has DNA-AP lyase activity. The protein sequence and encoding cDNA sequence are provided in FIGS. 7A and 7B. OGG1 polymorphisms are shown in Table 8.

TABLE 8
SNPs

| | dbSNP | Protein | Codon | Amino acid |
|---|---|---|---|---|
| Function | allele | residue | position | position |
| nonsynonymous | A | Thr [T] | 1 | 27 |
| contig reference | C | Pro [P] | 1 | 27 |
| nonsynonymous | T | Ser [S] | 1 | 85 |
| contig reference | G | Ala [A] | 1 | 85 |
| synonymous | A | Lys [K] | 3 | 98 |
| contig reference | G | Lys [K] | 3 | 98 |
| nonsynonymous | A | Gln [Q] | 2 | 229 |
| contig reference | G | Arg [R] | 2 | 229 |
| nonsynonymous | T | Val [V] | 2 | 288 |
| contig reference | C | Ala [A] | 2 | 288 |
| nonsynonymous | C | Thr [T] | 2 | 320 |
| contig reference | G | Ser [S] | 2 | 320 |
| nonsynonymous | A | Asn [N] | 1 | 322 |
| contig reference | G | Asp [D] | 1 | 322 |
| synonymous | T | Leu [L] | 1 | 323 |
| contig reference | C | Leu [L] | 1 | 323 |
| nonsynonymous | G | Cys [C] | 2 | 326 |
| contig reference | C | Ser [S] | 2 | 326 |
| synonymous | G | Ser [S] | 3 | 326 |
| contig reference | C | Ser [S] | 3 | 326 |

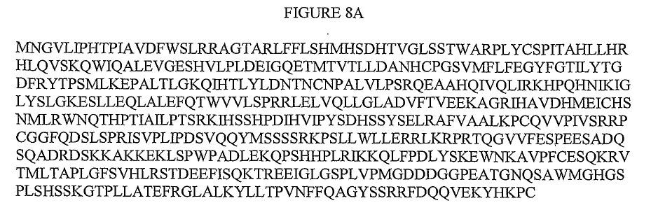

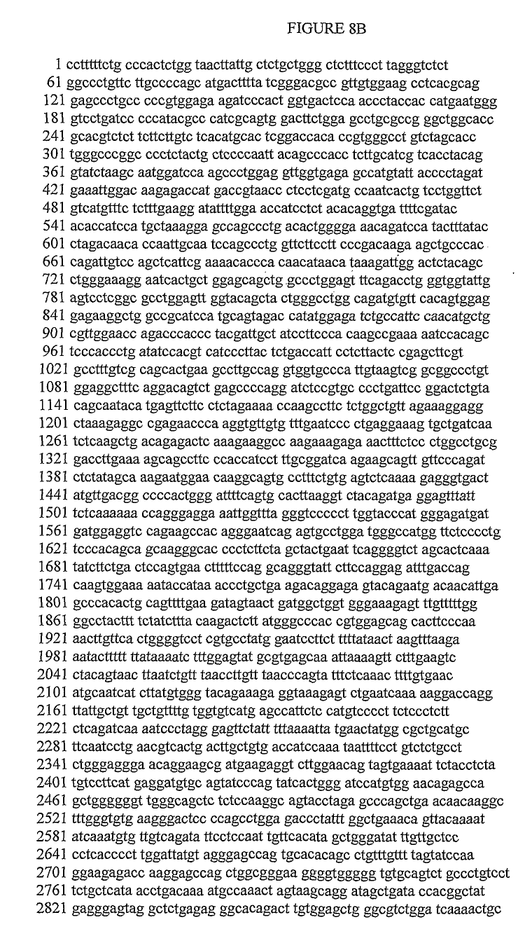

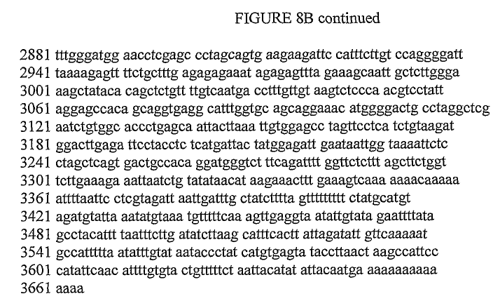

DCLRE1b: a protein containing a DNA repair metallo-beta-lactamase domain. It has a region of low similarity to a region of DNA cross-link repair protein (mouse Dclre1a), which is involved in repair of interstrand DNA cross-links. The protein sequence and encoding DNA sequence are provided in FIGS. 8A and 8B. DCLRE1b polymorphisms are shown in Table 9.

TABLE 9
SNPs

| | dbSNP | Protein | Codon | Amino acid |
|---|---|---|---|---|
| Function | allele | residue | position | position |
| nonsynonymous | T | Tyr [Y] | 1 | 61 |
| contig reference | C | His [H] | 1 | 61 |
| nonsynonymous | T | Phe [F] | 2 | 65 |
| contig reference | C | Ser [S] | 2 | 65 |
| nonsynonymous | A | Met [M] | 1 | 72 |
| contig reference | C | Leu [L] | 1 | 72 |
| synonymous | C | His [H] | 3 | 78 |
| contig reference | T | His [H] | 3 | 78 |
| nonsynonymous | A | His [H] | 2 | 81 |
| contig reference | C | Pro [P] | 2 | 81 |

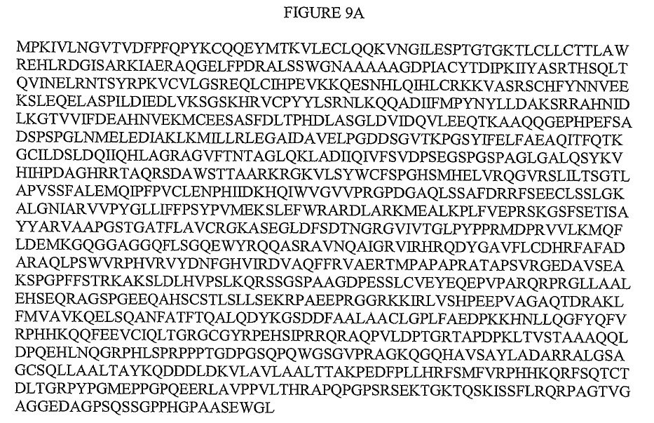

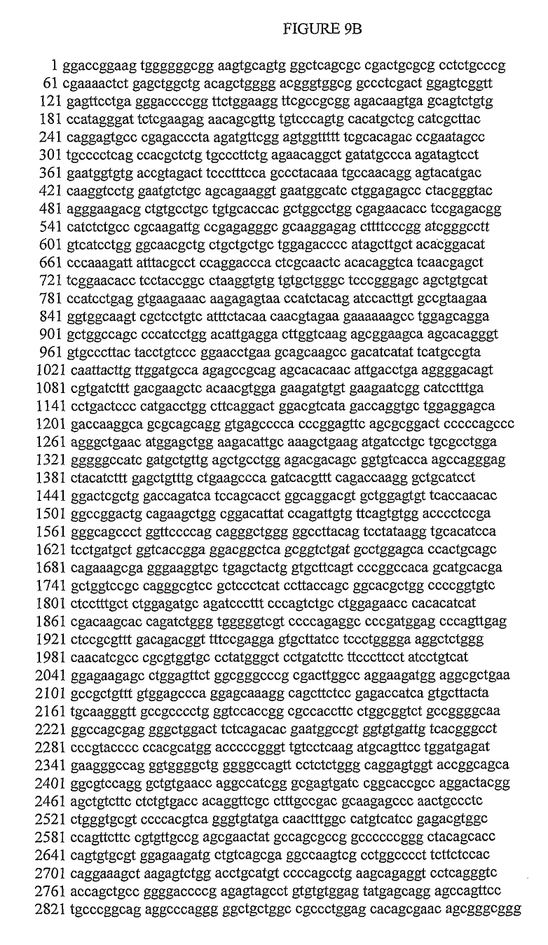

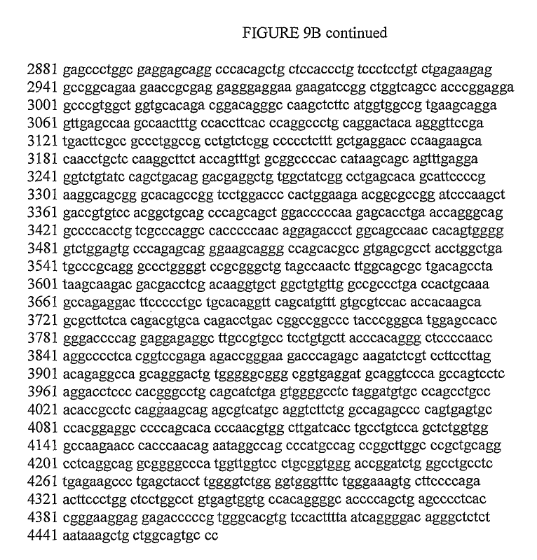

RTEL1: Protein with high similarity to regulator of telomere length (mouse Rtel1), which is a DNA helicase-like protein that regulates telomere length and chromosome stability. The protein sequence and encoding cDNA sequence are provided in FIGS. 9A and 9B. RTEL1 polymorphisms are shown in Table 10.

TABLE 10

| | dbSNP | Protein | Codon | Amino acid |
|---|---|---|---|---|
| Function | allele | residue | position | position |
| nonsynonymous | G | Ser [S] | 2 | 124 |
| contig reference | A | Asn [N] | 2 | 124 |
| synonymous | A | Lys [K] | 3 | 134 |
| contig reference | G | Lys [K] | 3 | 134 |
| synonymous | A | Ser [S] | 3 | 262 |
| contig reference | G | Ser [S] | 3 | 262 |
| synonymous | C | Gly [G] | 3 | 293 |
| contig reference | T | Gly [G] | 3 | 293 |
| nonsynonymous | T | Asn [N] | 3 | 659 |
| contig reference | G | Lys [K] | 3 | 659 |
| nonsynonymous | A | Ile [I] | 3 | 660 |
| contig reference | G | Met [M] | 3 | 660 |
| synonymous | C | Asp [D] | 3 | 664 |
| contig reference | T | Asp [D] | 3 | 664 |
| synonymous | A | Ala [A] | 3 | 758 |
| contig reference | G | Ala [A] | 3 | 758 |
| synonymous | C | Pro [P] | 3 | 848 |
| synonymous | C | Pro [P] | 3 | 848 |
| contig reference | T | Pro [P] | 3 | 848 |
| contig reference | T | Pro [P] | 3 | 848 |
| nonsynonymous | C | Asp [D] | 3 | 870 |
| contig reference | A | Glu [E] | 3 | 870 |
| synonymous | T | Pro [P] | 3 | 887 |
| contig reference | C | Pro [P] | 3 | 887 |
| synonymous | T | Ser [S] | 3 | 925 |
| contig reference | C | Ser [S] | 3 | 925 |
| synonymous | T | Phe [F] | 3 | 928 |
| contig reference | C | Phe [F] | 3 | 928 |
| synonymous | T | Leu [L] | 3 | 935 |
| contig reference | C | Leu [L] | 3 | 935 |
| nonsynonymous | A | Ser [S] | 1 | 951 |
| contig reference | G | Gly [G] | 1 | 951 |
| synonymous | A | Thr [T] | 3 | 1010 |
| contig reference | G | Thr [T] | 3 | 1010 |
| nonsynonymous | C | His [H] | 3 | 1042 |
| nonsynonymous | C | His [H] | 3 | 1042 |
| contig reference | A | Gln [Q] | 3 | 1042 |
| contig reference | A | Gln [Q] | 3 | 1042 |
| synonymous | A | Arg [R] | 1 | 1138 |
| contig reference | C | Arg [R] | 1 | 1138 |

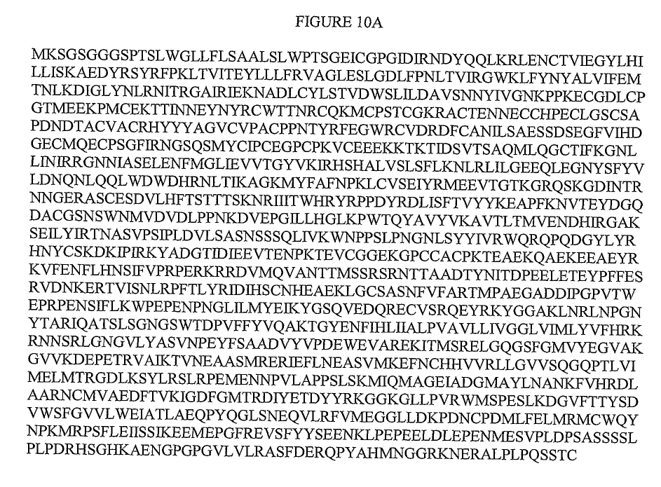

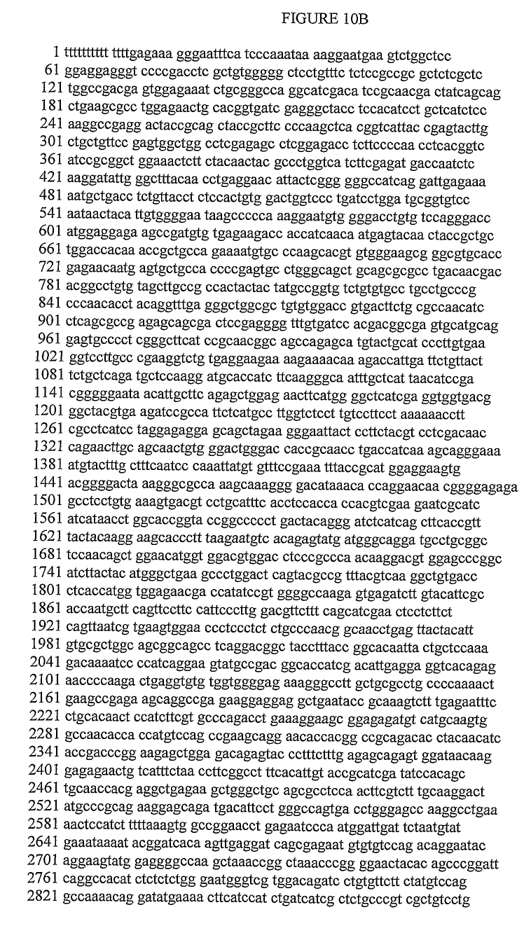

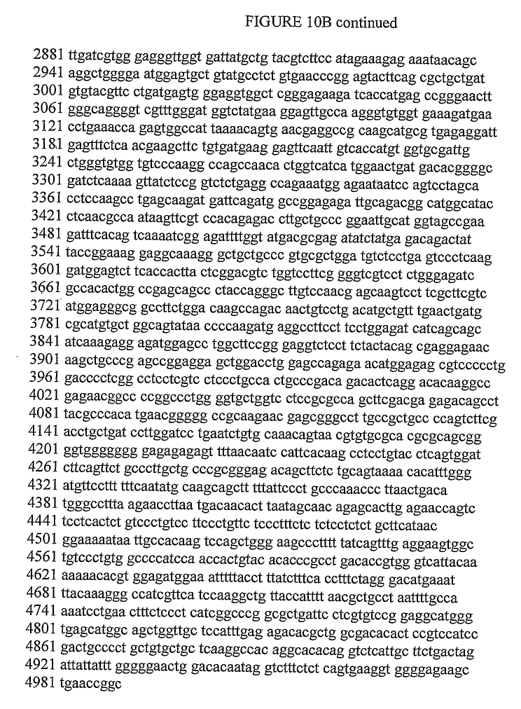

IGFR1: (insulin-like growth factor 1 receptor), which mediates IGF-1 stimulated cell proliferation and inhibits apoptosis. The protein sequence and encoding DNA sequence are provided in FIGS. 10A and 10B. IGFR1 polymorphisms are shown in Table 11.

TABLE 11
SNPs:

| | dbSNP | Protein | Codon | Amino acid |
|---|---|---|---|---|
| Function | allele | residue | position | position |
| synonymous | A | Val [V] | 3 | 562 |
| contig reference | G | Val [V] | 3 | 562 |
| synonymous | T | Pro [P] | 3 | 564 |
| contig reference | C | Pro [P] | 3 | 564 |
| synonymous | T | Thr [T] | 3 | 766 |
| contig reference | C | Thr [T] | 3 | 766 |
| synonymous | A | Glu [E] | 3 | 1043 |
| contig reference | G | Glu [E] | 3 | 1043 |

### Example 6

### Assessment of siRNA Efficacy in Preventing Production of Infectious Viral Particles

siRNAs targeting APEX1, DDB2, PMS2L1, POLE and POLI were tested for efficacy in preventing production of infectious viral particles. Briefly, HeLa P4/R5 cells were transfected with siRNAs targeting the above genes. The following day, cells were infected with HXB2 HIV. Four days after infection, a time point at which the virus has had an opportunity to infect cells and generate progeny virus which are released to the media, the viral supernatants were collected and used to infect freshly plated HeLa P4/R5 cells. Two days following infection, these cells were assessed for β-galactosidase expression as described above. A decrease in β-galactosidase activity in this assay signals that the levels of infectious HIV particles produced in cells treated with a particular siRNA are reduced, thus verifying that the decreased in HIV infection observed with the virus in Example 1 is owing to a direct effect on the viral life cycle and not to an effect on transcription of the β-galactosidase reporter gene or an indirect effect on cell metabolism.

### Results:

PMS2L1 siRNAs strongly inhibited production of infectious HIV, giving a greater than 80% reduction in the viral reinfection assay. POLE siRNAs resulted in more than 40% reduction in viral particle formation. APEX1 and DDB2 resulted in more than 30% reduction in viral particle formation. POLI resulted in 28% reduction in viral particle formation.

Other embodiments are within the scope of the following claims. All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. All such variations apparent to those skilled in the art are deemed to be within the spirit, scope and concept of the invention as defined by the appended claims.

1. A method of identifying a host cellular protein involved in HIV infection comprising the step of measuring the ability of a siRNA library targeting different host cell factors to inhibit HIV infection, wherein measuring the ability of a siRNA library to inhibit HIV infection further comprises:
transfecting human cells with the siRNA library targeting different cell factors;
infecting the transfected cells with HIV; and
assaying for viral infection to determine whether siRNA-mediated downregulation of host cell factors inhibits HIV infection.

2. The method of claim 1, wherein said siRNA library comprises at least 244 different siRNA's targeting a different host cellular protein not previously associated with HIV infection.

3. The method of claim 2, wherein the host cellular proteins are one or more components of a DNA repair pathway.

4. (canceled)

5. An assay for identifying a compound as an HIV inhibitor comprising the steps of:
identifying a compound that downregulates or otherwise inhibits the activity or expression of a target protein that is a component of a DNA repair pathway of a human cell; and
determining the ability of said compound to inhibit HIV.

6. The assay of claim 5, wherein the target protein is selected from the group consisting of: PMS2L1; ERCC3; RAD52; POLI; TNP1; POLL; CENPF; MSH6; NEIL2; BTG2; DDB2; DCLRE1b; RTEL1; ADPRT (PARP1); RAD51C; POLE; SMC6L1; APEX1; TAF2; OGG1; RUVBL2; RECQL4; TOP2A; ERCC3; RPA2; HMG4L; RBBP8; MLH1; MUS81; MSH4; IGF1R; XRCC4; RAD23B; ANKRD17; NTHL1; POLH; WDR33; DCLRE1A, and PMS1 and homologs.

7. (canceled)

8. The assay of claim 5 wherein the ability of said compound to inhibit HIV is determined comprising the steps of:
contacting the one or more components of a DNA repair pathway of a human cell with a noncircularized HIV DNA in the presence of a test compound;
contacting the or more components of a DNA repair pathway of a human cell with a noncircularized HIV DNA in the absence of a test compound; and
determining the effect of the test compound on HIV integration as measured by the amount of circularization.

9. The method of claim 8, wherein the one or more components of a DNA repair pathway of a human cell is a nucleic acid molecule encoding a polypeptide selected from the group consisting of:
PMS2L1; POLI; TNP1; POLL; CENPF; MSH6; NEIL2; BTG2; DDB2; DCLRE1b; RTEL1; RAD51C; POLE; SMC6L1; APEX1; TAF2; OGG1; RUVBL2; RECQL4; TOP2A; ERCC3; RPA2; HMG4L; RBBP8; MLH1; MUS81; MSH4; IGF1R; RAD23B; ANKRD17; NTHL1; POLH; WDR33; DCLRE1A; PMS1; and homologs thereof.

10. A method of treating HIV infection comprising decreasing the expression or activity of DNA repair pathway component selected from the group consisting of PMS2L1; POLI; TNP1; POLL; CENPF; MSH6; NEIL2; BTG2; DDB2; DCLRE1b; RTEL1; RAD51C; POLE; SMC6L1; APEX1; TAF2; OGG1; RUVBL2; RECQL4; TOP2A; ERCC3; RPA2; HMG4L; RBBP8; MLH1; MUS81; MSH4; IGF1R; RAD23B; ANKRD17; NTHL1; POLH; WDR33; DCLRE1A; and PMS1 in a patient in need thereof.
